SEQUENCE LISTING (24 sequences)

SEQ ID NO: 1 - DNA, 26 bp, Artificial Sequence
  Description of Artificial Sequence Primer
gtgctgcaggtgtaaacttgtaccag

SEQ ID NO: 2 - DNA, 28 bp, Artificial Sequence
  Description of Artificial Sequence Primer
cacggatccggtagcagcggtagagttg

SEQ ID NO: 3 - DNA, 21 bp, Artificial Sequence
  Description of Artificial Sequence Primer
gcatgtgctacttcaccaacg

SEQ ID NO: 4 - DNA, 29 bp, Artificial Sequence
  Description of Artificial Sequence Primer
cacctgcagatcccgcggtacgccacctc

SEQ ID NO: 5 - DNA, 21 bp, Artificial Sequence
  Description of Artificial Sequence Primer
gcatgtgctacttcaccaacg

SEQ ID NO: 6 - DNA, 28 bp, Artificial Sequence
  Description of Artificial Sequence Primer
cacctgcagtgcggagctccaactggta

SEQ ID NO: 7 - DNA, 21 bp, Artificial Sequence
  Description of Artificial Sequence Primer
ttcctgtggcagcctaagagg

SEQ ID NO: 8 - DNA, 21 bp, Artificial Sequence
  Description of Artificial Sequence Primer
gtttcttggagcaggttaaac

SEQ ID NO: 9 - DNA, 21 bp, Artificial Sequence
  Description of Artificial Sequence Primer
gaagcaggataagtttgagtg

SEQ ID NO: 10 - DNA, 21 bp, Artificial Sequence
  Description of Artificial Sequence Primer
ggttgctggaaagatgcatct

SEQ ID NO: 11 - DNA, 21 bp, Artificial Sequence
  Description of Artificial Sequence Primer
agttcctggaaagactcttct

SEQ ID NO: 12 - DNA, 21 bp, Artificial Sequence
  Description of Artificial Sequence Primer
ggttgctggaaagacgcgtcc

SEQ ID NO: 13 - DNA, 22 bp, Artificial Sequence
  Description of Artificial Sequence Primer
acgtttcttggagtactctacg

SEQ ID NO: 14 - DNA, 20 bp, Artificial Sequence
  Description of Artificial Sequence Primer
ccgctgcactgtgaagctct

SEQ ID NO: 15 - DNA, 21 bp, Artificial Sequence
  Description of Artificial Sequence Primer
cccagcacgtttcttggagct

SEQ ID NO: 16 - DNA, 20 bp, Artificial Sequence
  Description of Artificial Sequence Primer
ccgctgcactgtgaagctct

SEQ ID NO: 17 - DNA, 19 bp, Artificial Sequence
  Description of Artificial Sequence Primer
cttgcagcaggataagtat

SEQ ID NO: 18 - DNA, 20 bp, Artificial Sequence
  Description of Artificial Sequence Primer
ccgctgcactgtgaagctct

SEQ ID NO: 19 - DNA, 27 bp, Artificial Sequence
  Description of Artificial Sequence Primer
ggaagcttgatccccctgaggtgaccg

SEQ ID NO: 20 - DNA, 27 bp, Artificial Sequence
  Description of Artificial Sequence Primer
ggggatccccagtgcttgaggagcggc

SEQ ID NO: 21 - DNA, 28 bp, Artificial Sequence
  Description of Artificial Sequence Primer
ggaagcttgaggcccaagagccaatcca

SEQ ID NO: 22 - DNA, 26 bp, Artificial Sequence
  Description of Artificial Sequence Primer
ggggatccgccagaacgcagagactt

SEQ ID NO: 23 - DNA, 26 bp, Artificial Sequence
  Description of Artificial Sequence Primer
gtgaagctttccccgcagagaattac

SEQ ID NO: 24 - DNA, 27 bp, Artificial Sequence
  Description of Artificial Sequence Primer
cacctgcagtcactcacctcggcgctg

(19) United States
(12) Patent Application Publication
(10) Pub. No.: US 2004/0053272 A1
(43) Pub. Date: Mar. 18, 2004

(54) Methods of constructing a model of cellular development and differentiation using homozygous stem cell systems, methods of assessing and cataloging proteins expressed therein, cDNA libraries generated therefrom, and materials and methods using same

(76) Inventors: Steven Chien-Wen Huang, Germantown, MD (US); Hua (Helen) Lin, North Potomac, MD (US)

Correspondence Address: SHANKS & HERBERT, 1033 N. FAIRFAX STREET, SUITE 306, ALEXANDRIA, VA, 22314, US

(21) Appl. No.: 10/377,033

(22) Filed: Mar. 3, 2003

Related U.S. Application Data
(60) Provisional application No. 60/361,065, filed on Mar. 1, 2002.

(51) Int. Cl. C12Q 1/68; G01N 33/53; G01N 33/567; C12N 5/08

(52) U.S. Cl. 435/6; 435/7.2; 435/455; 435/366

(57) ABSTRACT

The present invention relates to materials and methods for the quantitative and qualitative analysis of gene expression during targeted differentiation of isolated homozygous stem cells, and to materials and methods for accumulating sequence tags sampled from a population of expressed genes. More particularly, the present invention relates to a method for constructing a model of cellular development and differentiation comprising the steps of: (1) producing a homogenous population of homozygous stem (HS) cells; (2) directing the differentiation of the HS cells to arrive at desired mature (somatic) cells, group of cells or tissue; (3) periodically sampling HS cells undergoing differentiation to extract total cellular RNA produced at various sampling stages; (4) isolating mRNA from the total cellular RNA; and (5) constructing a cDNA library representative of proteins produced and expressed at various stages of differentiation, over the course of time. The present invention further relates to sequencing and cataloguing isolated mRNAs and cDNAs at various stages of differentiation of HS cells, producing and cataloguing isolated polypeptides sequences encoded by same, and screening the cDNA libraries. In vitro and in vivo cultures of homozygous stem cell are also described; such cultures find utility both as models of differentiation as well as sources of differentiated tissues and organs.

### CROSS REFERENCE TO RELATED APPLICATIONS

The materials and methods described in the present invention are useful in combination with materials and methods described in U.S. patent application Ser. No. 09/997,240, filed Nov. 30, 2001, entitled “Isolated Homozygous Stem Cells, Differentiated Cells Derived Therefrom, And Methods of Making And Using Same”, which claims the benefit of U.S. Provisional Application Serial No. 60/253,943, filed Nov. 30, 2000, and U.S. patent application Ser. No.10/032,495, filed Jan. 2, 2002, entitled “A Method For Producing A Population Of Homozygous Stem Cells Having A Pre-Selected Immunophenotype And/Or Genotype, Cells Suitable For Transplant Derived Therefrom, And Materials And Methods Using Same”, which claims the benefit of U.S. Provisional Application Serial No. 60/258,881, filed Jan. 02, 2001. The entire content of these applications is hereby incorporated by reference herein.

### TECHNICAL FIELD AND INDUSTRIAL APPLICABILITY OF INVENTION

The present invention relates to materials and methods for the quantitative and qualitative analysis of gene expression during targeted differentiation of isolated homozygous stem cells, and to materials and methods for accumulating sequence tags sampled from a population of expressed genes. More particularly, the present invention relates to a method for constructing a model of cellular development and differentiation comprising the steps of: (1) producing a homogenous population of homozygous stem (HS) cells; (2) directing the differentiation of the HS cells to arrive at desired mature (somatic) cells, group of cells or tissue; (3) periodically sampling HS cells undergoing differentiation to extract total cellular RNA produced at various sampling stages; (4) isolating mRNA from the total cellular RNA; and (5) constructing a cDNA library representative of proteins produced and expressed at various stages of differentiation, over the course of time.

The present invention further relates to sequencing and cataloguing isolated mRNAs and cDNAs at various stages of differentiation of HS cells, producing and cataloguing isolated polypeptides sequences encoded by same, and screening the cDNA libraries. In vitro and in vivo cultures of homozygous stem cell are also described; such cultures find utility both as models of differentiation as well as sources of differentiated tissues and organs.

### BACKGROUND OF THE INVENTION

The present invention may be used to genetically characterize cellular development, disease states, and a host of other physiological states associated with differential gene expression during various phases of cellular differentiation. Additionally, normal differentiation processes can be compared to differentiation processes in disease states to diagnose and develop therapeutic and prophylactic treatments for diseases, such as genetic diseases, cancer, and developmental anomalies.

The human genome is estimated to contain from about 30,000 to about 100,000 genes, of which only about 15-30% are active in any given tissue. Moreover different genes are active in different types of cells and tissues, i.e. there is differential expression of the total genes found in the human genome in various different types of cells and tissues. Hence, many of the most fundamental questions of human physiology can be answered by a basic understanding of which genes are expressed and at what relative abundance in different cells over the course of time.

Despite the fact that only about 15-30% of the human genome is active in any given tissue, the number of active genes in any given tissue is still so large that tracking changes in expression patterns of such activated genes using currently available molecular techniques a difficult task. Examples of current techniques useful in the analysis of differential gene expression include, but are not limited to, hybridization of gene products to micro arrays (see, Marx, J., Science (2000), 289(5485): 1670-1672, incorporated by reference herein) and direct sequence analysis. Lower resolution techniques include, differential display, indexing, subtraction hybridization, or DNA fingerprinting techniques. See, Vos et al, Nucleic Acids Research (1995), 23: 4407-4414; Hubank et al, Nucleic Acids Research (1994), 22: 5640-5648; Lingo et al, Science (1992), 257: 967-971; Erlander et al, WO95/13369; McClelland et al, U.S. Pat. No. 5,437,975; Unrau et al, Gene (1994), 145: 163-169; and, Geng et al, BioTechniques (1998), 25: 434-438, which are incorporated by reference herein. Higher resolution analysis is currently carried out on subsets of cDNA clones identified by the application of such techniques. See, Linskens et al, Nucleic Acids Research (1995), 23: 3244-3251, incorporated by reference herein.

Moreover, known techniques to date allow the analysis of differential expression of only a few known genes at a time using standard molecular biology techniques such as PCR, northern blot analysis, or other types of DNA probe analysis such as in situ hybridization. Thus, clearly there is a need in the art for a system that allows the analysis of differential expression of various known and unknown genes in differentiating and differentiated cells and/or tissues, and the assignment of a sequence tag to an expressed gene. The availability of such a system would find immediate application in medical and scientific research, diagnostics, drug discovery, and genetic analysis in a host of applied fields.

Studies of the number and types of genes whose synthesis is induced or otherwise regulated during developmental processes, such as cell activation, differentiation, aging, viral transformation, morphogenesis, and division have been pursued for many years using a variety of methodologies. One of the earliest methods was to compare the proteins made in a given cell, tissue, organ system, or even organism both prior to and subsequent to the differentiation process of interest. Such comparisons were typically made using 2-dimensional gel electrophoresis, wherein each protein could be identified and quantified as a discrete signal. However, in order to positively identify each signal, each discrete signal must be excised from the membrane and subjected to protein sequence analysis typically using Edman degradation. Unfortunately, most of the signals are present in quantities too small to obtain a reliable sequence, and many of those signals contained more than one discrete protein. An additional difficulty is that many of the proteins were blocked at the amino-terminus, further complicating the sequencing process.

Analyzing differentiation at the gene transcription level overcomes many of these disadvantages and drawbacks, since the power of recombinant DNA technology allows amplification of signals containing very small amounts of material. RNA extracted from the biological sample can be used to construct a cDNA library, which in turn provides a catalogue of proteins expressed by the sample at a particular stage of development.

Thus, in response to a clear need in the art, the present invention provides models of cellular development and differentiation derived from populations of isolated homozygous stem cells and differentiated cells arising therefrom. The present invention further provides methods for constructing such models involving the steps of periodic sampling of HS cells undergoing differentiation to extract total cellular RNA produced at various sampling stages, and isolating mRNA from the total cellular RNA to construct a cDNA library.

The construction of cDNA libraries is conventional in the art, and the present invention incorporates by reference U.S. Pat. No. 5,846,721 (Soares et al., 1998), and U.S. Pat. No. 6,136,537 (Macevicz, 2000) describing such techniques. Additionally, the present invention relates to sequencing and cataloguing isolated mRNAs and cDNAs at various stages of differentiation of HS cells, producing and cataloguing isolated polypeptides sequences encoded by same, and screening the cDNA libraries to identify the quantity and quality of proteins encoded thereby.

### SUMMARY OF THE INVENTION

It is an object of the present invention to provide a method for constructing a model of cellular development and differentiation using isolated homozygous stem (HS) cells, or progenitor or other cells derived therefrom. In one embodiment, the method entails the steps of: (a) creating a population of isolated HS cells; (b) producing a desired progenitor or other mature (somatic) cell, group of cells or tissue type by directing the differentiation of said HS cells under controlled conditions; (c) sampling said HS cells undergoing directed differentiation intermittently, where the sampling period will vary depending on the individual tissue developmental process; (d) extracting cellular RNA, and then isolating mRNA from said sampled HS cells; and, (e) constructing a cDNA library from said isolated mRNA.

In one embodiment, the model of cellular development comprises a population of HS cells cultured in vitro. In another embodiment, the model of cellular development comprises a population of HS cells cultured in vivo. Such HS cultures find utility both as models of differentiation and sources of differentiated tissues and organs.

The present invention utilizes the methods of producing HS cells described in the co-pending U.S. patent application Ser. Nos. 09/997,240, and 10/032,495, incorporated by reference herein. Such methods provide HS cells derived from blastocyst-like masses mitotically created by: (a) fusing two oocytes or two spermatids; (b) preventing the extrusion of the second polar body during oogenesis; (c) allowing the extrusion of the second polar body and spontaneous genomic self-replication in appropriate conditions; or, (d) transferring two haploid egg or sperm nuclei into an enucleated oocyte. Additionally, screening for stem cells that are homozygous is performed using genotyping when method (a) or (d) are used. HS cells may also be derived from stemplasms.

The present invention further utilizes methods of making a desired cell, group of cells or tissue type described in the co-pending U.S. patent application Ser. Nos. 09/997,240, and 10/032,495, incorporated by reference herein. Such methods involve directing the differentiation of isolated HS cells under suitable conditions using factors in vivo or in vitro, so as to arrive at the desired cell, group of cells, or tissue type, also disclosed in the above-referenced application fully incorporated by reference herein. Exemplary tissues include, but are not limited to, tissues of the epithelium, connective tissue, muscle tissue or nervous tissue.

Further, illustrative types of epithelial cells include but are not limited to keratinizing epithelial cells; wet-stratified barrier epithelia; lining epithelial cells; exocrine-secreting epithelial cells; endocrine-secreting epithelial cells; extracellular matrix-secreting epithelial cells; absorptive epithelial cells, such as those of the gut, exocrine glands, and urogenital tract; and contractile epithelial cells. Illustrative types of connective tissue cells include but are not limited to extracellular matrix-secreting cells; cells specialized for metabolism and storage; and circulating cells of the blood and immune systems. Illustrative types of muscle cells include but are not limited to contractile cells and ciliated cells with propulsive function. Illustrative types of nervous or sensory cells include but are not limited to sensory transducers; autonomic neurons; supporting cells of sense organs and of peripheral neurons; and neurons and glial cells of central nervous system. Illustrative types of reproductive cells include but are not limited to germ cells and nurse cells.

It is a further object of the present invention to isolate and extract mRNA from isolated HS cells at various stages of directed differentiation in vivo, and/or in vitro using techniques known in the art. Such mRNA may also include a functional promotor sequence at the 5′ end, and/or at least one copy of each mRNA may be present in a recombinant RNA vector. Additionally, it is an object of the present invention to genetically engineer cDNA molecules using mRNA extracted and isolated from isolated HS cells at various stages of in vivo, and/or in vitro differentiation, where at least one copy of each engineered cDNA molecules is present in a recombinant DNA vector.

It is another object of the present invention to make oligonucleotides complementary to mRNA, or to cDNA that is complementary to said mRNA, that is extracted and isolated from isolated HS cells at various stages of directed differentiation, comprising at least 10 consecutive nucleotides 65-100% identical to said mRNA and/or cDNA. Additionally, the present invention discloses a method of identifying genetic material encoding various genes involved in the differentiation of isolated HS cells into a desired cell, group of cells, or tissue type, that entails contacting a sample of genetic material extracted from individual HS cells with said oligonucleotides under hybridizing conditions, and detecting the formation of a duplex comprising said oligonucleotide and said genetic material present in said sample.

A further object of the present invention is to isolate polypeptide sequences encoded by mRNA molecules extracted and isolated from isolated HS cells at various stages of directed differentiation.

Another object of the present invention is to provide methods of identifying genetic material encoding various genes involved in the differentiation of isolated HS cells into a desired cell, group of cells, or tissue type. Methods entail isolating genetic material from differentiating HS cells as a sample, contacting said sample with an oligonucleotide under hybridizing conditions, and detecting the formation of a duplex comprising said oligonucleotide and said genetic material present in said sample.

Another embodiment of the present invention involves the construction of a series of cDNA libraries, each representing to proteins expressed over the course of HS cell development and differentiation under one or more differentiation conditions. The invention further provides for the generation of a searchable database comprised of the gene and protein sequence data represented by the series of cDNA libraries.

### BRIEF DESCRIPTION OF THE DRAWING

FIG. 1 table of amino acids and corresponding nucleic acid sequences;

FIG. 2 flow chart depicting a preferred method of developing hs cells from a non-fertilized post-meiosis i diploid germ cell;

FIG. 3 a schematic representation of spermatogenesis and oogenesis, showing the difference in phases of mitosis and meiosis in males and females;

FIG. 4A C57/DBA2 mouse;

FIG. 4B superovulated oocytes-derived blastocysts-like masses;

FIG. 4C representative colony I;

FIG. 4D representative colony II;

FIG. 4E results of genotyping experiment;

FIG. 4F results immunotyping experiment;

FIG. 5A photo of an isolated inner cell mass growing on feeder layers derived from mouse HS cells;

FIG. 5B photo depicting the development of a morula-like mass derived from human homozygous post-meiosis I diploid oocytes;

FIG. 5C photo of an early blastocyst-like mass derived from human homozygous post-meiosis I diploid oocytes;

FIG. 5D photo of a blastocyst-like mass revealing the inner cell mass derived from human homozygous post-meiosis I diploid oocytes;

FIG. 6 an exemplary list of PCR primers for amplification of HLA class II alleles; and

FIG. 7 a list of exemplary restriction endonucleases for genotyping of HLA class II alleles.

### DETAILED DESCRIPTION OF A PREFERRED EMBODIMENT OF THE INVENTION

All references cited herein are hereby incorporated by reference in their entirety. Nothing herein is to be construed as an admission that the invention is not entitled to antedate such disclosure by virtue of prior invention or that the prior art provides an enabling or adequate disclosure. Throughout this description, the preferred embodiments and examples shown should be considered as exemplary, rather than as limitations on the present invention.

### A. DEFINITIONS

In the context of the present invention, the following definitions apply.

“Differentiation” is a highly regulated process that cells undergo as they mature into normal functional cells. Differentiated cells have distinctive characteristics, perform specific functions and are less likely to divide. Conversely, undifferentiated cells are rapidly dividing immature, embryonic or primitive cells having a nonspecific appearance with multiple nonspecific activities and functions.

As used herein, the term “stem cell” refers to a relatively undifferentiated cell that actively divides and cycles, giving rise upon proper stimulation to a lineage of mature, differentiated, functional cells. The defining properties of a stem cell include: (a) it is not itself terminally differentiated; (b) it can divide without limit for the lifetime of the animal; and (c) when it divides, each daughter has a choice of remaining a stem cell or embarking on a course that leads irreversibly to terminal differentiation. Those stem cells that are initially unrestricted in their capabilities (i.e., capable of giving rise to several types of differentiated cell) are called “pluripotent”. Current sources of pluripotent cells include embryonic (ES) stem cells, embryonic carcinoma (EC) cells, cells generated from somatic cloning, teratomas and teratocarcinomas.

Progenitor cell lines, each capable of producing cells from one of the three germ layers, i.e. the endoderm, mesoderm and ectoderm, are referred to in the present application as “multi-potent”. While each progenitor cell line is not terminally differentiated and can continue to divide for the lifetime of an animal, it is considered to be committed to different tissues or cells from only one type of embryonic layer. Therefore, particular progenitor cell lines may be differentiated into bone, cartilage, smooth muscle, striated muscle and hematopoietic cells (mesoderm); liver, primitive gut, and respiratory epithelium (endoderm); or, neurons, glial cells, hair follicles and tooth buds (ectoderm). The term “progenitor cells” hence may be used synonymously with “multi-potent stem cells” or “precursor cells”. Such progenitor cells lines, which are created by the directed differentiation of HS cells in vivo (where the term “in vivo” includes differentiation induced by encapsulating said HS cells in an isogenic or allogeneic animal to generate stemplasms from such encapsulated cells) or in vitro, can be maintained in culture as permanent cell lines.

A “teratoma” is a naturally occurring spontaneous mass of abnormal cells containing many types of differentiated tissue, tissues derived from all three embryonic layers, such as bone, muscle, cartilage, nerve, tooth-buds, glandular epithelium, and so forth, mixed with undifferentiated stem cells that continually divide and generate yet more of these differentiated tissues.

A teratoma is a spontaneously formed neoplasm usually found in reproductive tissues, which contains cells from all the three embryonic germ layers. Further, it is characterized by unregulated growth. A “stemplasm” is a newly derived term used to describe a mass that develops upon the transplantation of HS cells into a host. Unlike teratomas, a stemplasm exhibits controlled growth, while still containing cells from all three embryonic germ layers. It can therefore be used as a means for the in vivo differentiation of the HS cells of the present invention.

A “teratocarcinoma” is secondary to a teratoma. Teratomas are largely benign; however if they become malignant, a teratocarcinoma develops and can be deadly to the host.

A “homozygous stem cell”, previously termed a “teratoma stem cell” or a “TS cell”, is an undifferentiated stem cell arising from a non-fertilized post-meiosis I diploid germ cell. Preferably, it is formed by preventing the extrusion of the second polar body during oogenesis (or “activation”), or allowing the extrusion of the second polar body and spontaneous genomic self-replication of the haploid oocyte in appropriate conditions. Homozygous stem (HS) cells are isolated cells generated from the inner cell mass of blastocyst-like masses that develop upon “mitotic activation” of non-fertilized post-meiosis I diploid germ cells, which can be accomplished by: (a) fusing two oocytes or two spermatids; (b) preventing the extrusion of the second polar body during oogenesis; (c) allowing the extrusion of the second polar body and spontaneous genomic self-replication in appropriate conditions; or, (d) transferring two haploid egg or sperm nuclei into an enucleated oocyte. Additionally, screening for stem cells that are homozygous is performed using genotyping when method (a) or (d) are used.

In mammalian development, cleavage produces a thin-walled hollow sphere, the “blastocyst”, with the embryo proper being represented by a mass of cells at one side, otherwise known as the “inner cell mass”. The blastocyst is formed before implantation and is equivalent to the “blastula”. The wall of the thin-walled hollow sphere is referred to as the “trophoblast”, which is the extra-embryonic layer of epithelium that forms around the mammalian blastocyst, and attaches the embryo to the uterus wall. The trophoblast forms the outer layer of the chorion, and together with maternal tissue will form the placenta.

In the context of the present invention, a “blastocyst-like mass” is different from a “blastocyst” (as used in the art) in that it is the product of a mitotically activated non-fertilized post-meiosis I germ cell.

As used herein, the term “mitotically activated” means acquiring the ability to undergo regular cell divisions mitotically, and includes both parthenogenetic activation of oocytes and androgenetic activation of spermatids.

The term “homozygous post-meiosis I diploid germ cells”, as used herein, means germ cells that are the stage of gametogenesis at which the cells contain two copies of either the paternal or maternal homologous chromosomes.

“Equivalent” is used when referring to two nucleotide sequences, wherein the two nucleotide sequences in question encode the same sequence of amino acids. When “equivalent” is used in referring to two peptides, it means that the two peptides will have substantially the same amino acid sequence (i.e. at least 70% homologous). When “equivalent” refers to a property, the property does not need to be present to the same extent (e.g., two peptides can exhibit different rates of the same type of enzymatic activity), but the properties are preferably substantially the same.

“Complementary,” when referring to two nucleotide sequences, means that the two sequences are capable of hybridizing, preferably with less than 25%, more preferably with less than 15%, even more preferably with less than 5%, most preferably with no mismatches between opposed nucleotides. Preferred hybridizing conditions (which are not limited to specific numbers of mismatches) are set forth in the Examples. The term “substantially” varies with the context as understood by those skilled in the relevant art and generally means at least 70%, preferably means at least 80%, more preferably at least 90%, and most preferably at least 95%. The phrase “substantially identical” includes complete identity as well as less than complete identity (e.g., of amino acid sequences or enzymatic activity) as established by the prior definition of “substantially.”

The term “isolated” as used herein refers to, e.g., a peptide, DNA, or RNA separated from other peptides, DNAs, or RNAs, respectively, and being found in the presence of (if anything) only a solvent, buffer, ion or other component normally present in a biochemical solution of the same. “Isolated” does not encompass either natural materials in their native state or natural materials that have been separated into components (e.g., in an acrylamide gel) but not obtained either as pure substances or as solutions. The phrase “replaced by” or “replacement” as used herein does not necessarily refer to any action that must take place but to the peptide that exists when an indicated “replacement” amino acid is present in the same position as the amino acid indicated to be present in a different formula (e.g., when leucine is present instead of valine).

### B. CREATION OF HOMOZYGOUS STEM CELLS OF THE PRESENT INVENTION

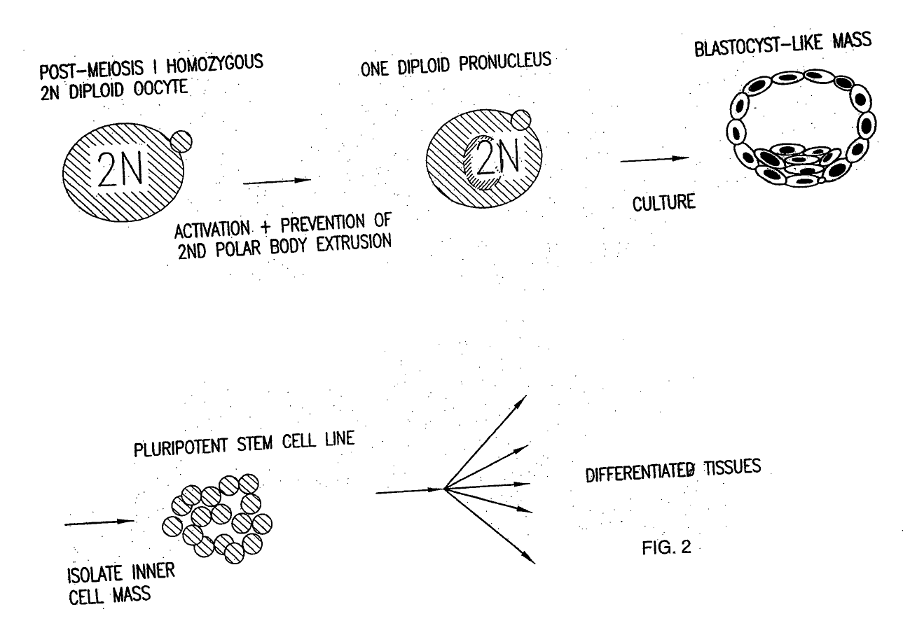

As described in detail in U.S. application Ser. No. 09/997,240, an HS cell is isolated from a blastocyst-like mass that develops upon the mitotic activation of a non-fertilized post-meiosis I diploid germ cell. FIG. 2 provides a flow chart, showing a preferred method of developing HS cells from a non-fertilized post-meiosis I diploid germ cell.

Germ cells develop into non-fertilized post-meiosis I diploid germ cells that, upon activation, produce blastocyst-like masses from which the HS cells of the present invention are derived. HS cells, and/or differentiated cells, of the present invention find utility in the diagnosis and/or treatment of diseases, include, but are not limited to implantation or transplantation to an affected individual in need of such therapy.

While homozygous post-meiosis I diploid germ cells may be obtained from the same individual or from an immunocompatible donor, in certain situations self-donors are preferred. However, in cases where the affected individual selected for therapy suffers from a genetic disease (i.e., a disease characterized by a lack of a crucial gene, either due to mutation or improper expression), it may be preferable to utilize a non-self donor. Alternatively, one skilled in the art of selection procedures may choose those self germ cells that display the desired genotype (e.g., cells lacking a flawed or mutated gene), those cells capable of expressing the deficient gene. Such selection techniques may also be used to avoid an immuno-incompatible genotype or phenotype for tissue transplant.

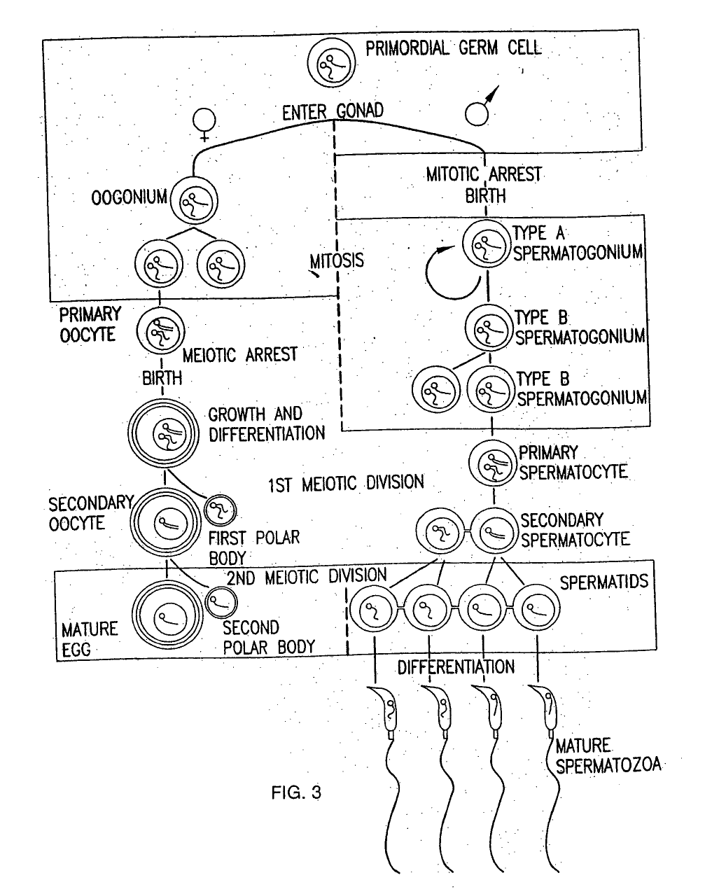

Methods for obtaining HS cells that are pre-selected for immunophenotype and genotype, and therapeutic and/or prophylactic uses of such cells are described in U.S. application Ser. No. 10/032,495 that is incorporated by reference herein. Briefly, HS cells that are homozygous for a HLA haplotype and a target gene can be derived and selected from: (a) fusing two oocytes or two spermatids; (b) preventing the extrusion of the second polar body during oogenesis; (c) allowing the extrusion of the second polar body and spontaneous genomic self-replication in appropriate conditions; or, (d) transferring two haploid egg or sperm nuclei into an enucleated oocyte. FIG. 3 provides a schematic representation of spermatogenesis and oogenesis, showing the difference in phases of mitosis and meiosis in males and females.

Oocytes useful in the context of the present invention may be obtained using any suitable method known in the art, or yet to be discovered. Human oocytes are typically harvested from the ovarian follicles of a donor individual and isolated from surrounding or adhering cells. To maximize yield, superovulation is induced in the donor individual. Superovulation may be induced by the administration of appropriate gonadotropins or gonadotropin analogues, administered either alone or in combination with clomiphene citrate (Barriere et al., Rev. Prat., 40(29):2689-93 (1990), incorporated by reference herein). In mice, an exemplary method involves the administration of pregnant mare's serum (PMS) to mimic follicle-stimulating hormone (FSH) and human chorionic gonadotropin (hCG) to mimic luteinizing hormone (LH). See Hogan et al., Manipulating the mouse embryo: A Laboratory Manual, 2^{nd} ed. Cold Spring Harbor Laboratory Press, 1994). Efficient induction of superovulation depends on several variables including, but not limited to, the age and weight of the female, the dose of gonadotropin, the time of administration, and the strain used.

Superinduction of ovulation and harvesting of oocytes are known in the art. For exemplary detailed mouse protocols, see Hogan et al., supra, pp. 130-132, the entire contents of which are hereby incorporated by reference. For example, Hogan describes the intraperitoneal administration of PMS and hCG, both resuspended from lyophilized powder in sterile 0.9% NaCl, to induce superovulation. Both PMS and hCG should be administered prior to the release of endogenous LH. The Hogan protocols, directed to the harvesting of oocytes from mice, can be routinely adapted for humans without undue experimentation.

Polyethylene glycol has also been shown to induce fusion of ovulated oocytes (see, e.g., Sekirina, G. G., Ontogenez., 16(6):583-8 (1985), and Gulyas, B. J., Dev. Biol., 101(1):246-50 (1984), incorporated by reference herein). Alternatively, Nogues et al., Zygote, 2(1):15-28 (1994), (incorporated by reference herein) describes the induction of oocyte fusion by inactivated Sendai virus, resulting in the production of “zygotes” or “oocyte fusion products (OFP)” that are able to undergo the first stages of embryonic development. For a review of oocyte fusion techniques, see Gulyas, B. J., Dev. Biol., 4:57-80 (1986), incorporated by reference herein.

Alternatively, preventing the extrusion of the second polar body from oocytes can generate HS cells. A detailed protocol for activation of mouse oocytes is described in Hogan et al. supra, pp.148-150, wherein harvested eggs with their cumulus cells attached are maintained in a solution of 7% ethanol in Dulbecco's PBS for 5 minutes, washed with medium, and incubated at 37° C. for 5 hours. The cumulus cells are subsequently removed by treatment with hyaluronidase. In nature, following the first meiotic division and separation of the first polar body, oocytes are arrested at metaphase II and can only undergo the second meiotic division when stimulated by a sperm. In vitro, the stimulation from the sperm can be mimicked by exposing oocytes to agents such as Ca⁺⁺ ionophore (A23187) or ethanol to trigger the continuation of meiosis II. Before the extrusion of the second polar body, karyokinesis (separation of chromosomes) and cytokinesis (division of cells) can be inhibited by agents including, but not limited to, 6-dimethylaminopurine (6-DMAP), or cytochalasin D, resulting in the activation of such diploid oocytes and subsequent formation of blastocyst-like masses. FIG. 2 depicts the products of activation.

In another embodiment, allowing the extrusion of the second polar body can be accomplished by exposing oocytes to Ca⁺⁺ ionophore (A23187) alone or followed by puromycin. The haploid oocytes can further undergo genomic self-replication without division, and the resulting diploid oocytes when incubated under appropriate conditions can form blastocyst like masses and may be used to derive HS cells. See, Taylor, A. S., et al., Hum. Reprod., 9(12):2389-97 (1994); and Kaufman, M. H. et al., J. Embryol. Exp. Morphol., 73:249-61 (1983).

Spermatids useful in the context of the present invention can be obtained using any suitable method known in the art or yet to be discovered, particularly those conventional in the field of in vitro fertilization. To create HS cells for use in a male, spermatids (meiosis II completed) are harvested and then induced to fuse. Spermatid fusion can be achieved using well-established standard techniques. For example, Asakura S, et al., Exp. Cell. Res., 181(2):566-73 (1989), incorporated by reference herein, teaches the use of a hypotonic medium to induce the fusion of a pair of spermatids and the eventual formation of a single acrosome (synacrosome). Alternately, secondary spermatocytes (meiosis I completed) can be activated using methods that are known in the art.

Homozygous post-meiosis I diploid germ cells can be harvested from a donor using conventional technology, particularly those techniques commonly used in the field of in vitro fertilization. See, for example, Jones et al., Fertil. Steril., 37(1):26-29 (1982), describing techniques for aspirating oocytes from human ovarian follicles; Lisek et al., Tech. Urol., 3(2):81-85 (1997), describing techniques for collecting sperm from the epididymis and testicle; Stice et al., Mol. Reprod. Dev., 38(1):61-8 (1994), and Takeuchi et al., Hum. Reprod., 14(5):1312-7 (1999), describing techniques for transplanting nuclear material of one donor to an enucleated oocyte of another. The entire contents of these references are hereby incorporated by reference herein.

Finally, as noted above, the isolated HS cell can be created from transferring two haploid germ cell nuclei to an enucleated oocyte. Specifically, two sperm or haploid egg nuclei can be transferred into an enucleated oocyte to create a non-fertilized diploid oocyte bearing the nuclear genetic information of the donor male or female in the oocyte cytoplasm. The donor nuclear material can be harvested and/or isolated using standard techniques conventional in the art. Likewise, the transfer step can be performed using techniques conventional in the art of in vitro fertilization (see U.S. Pat. No. 5,945,577, WO 98/07841, and see also, Stice and Takeuchi discussed above, as well as Wobus et al., Cells Tissues Organs, 166:1-5 (2000) incorporated by reference herein.

Genetic modifications may be introduced into HS cells by polynucleotide transfection techniques, including but not limited to, viral vector transfer, bacterial vector transfer, and synthetic vector transfer (e.g., via plasmids, liposomes and colloid complexes).

Methods for isolating ES cells from the inner cell mass of fertilized blastocysts are known in the art. Such methods may be adapted for isolating HS cells from the inner cell mass of blastocyst-like masses. For example, see Gardner et al., Curr. Op. Obst. Gyn., 11:307-311 (1999), U.S. Pat. No. 5,843,780 (Thomson et al.) and U.S. Pat. No. 5,905,042 (Stice et al.), the contents of which are incorporated by reference herein.

HS cells can be produced from any animal donor material and used in any animal system. Both human and non-human HS cells are contemplated by the present invention. Suitable veterinary applications include the generation of HS cells from and use in mammals, fish, reptiles, birds, and amphibians.

### C. METHODS FOR DIRECTED DIFFERENTIATION OF HS CELLS

HS cells can be induced to differentiate into various types of tissues originating from all three germ layers (endoderm, mesoderm, and ectoderm) including, but not limited to, skin, hair, nervous tissue, pancreatic islet cells, bone, bone marrow, pituitary gland, liver, bladder, and other tissues having diagnostic or therapeutic utility in animals, including humans as described in U.S. patent application Ser. Nos. 09/997,240 and 10/032,495, fully incorporated by reference herein.

One skilled in the art of differentiation techniques, particularly those developed for differentiation of ES cells and embryonic carcinoma (teratocarcinoma) cells, can induce a pluripotent HS cell to differentiate into a desired type/of tissue without undue experimentation.

The pluripotent isolated HS cells of the present invention can be differentiated into selected tissues for a variety of therapeutic uses including the in vitro culture of differentiated tissues for purposes of study, diagnostics, or for implantation into an individual. Preferably, HS cells will be used therapeutically in the individual that provided the donor material for HS formation. In females, this may be achieved by activation of post-meiosis I diploid oocytes, or by fusion two haploid oocytes; in males, this may be achieved by activation of secondary spermatocytes or fusion of spermatids, or by transferring sperm nuclei to enucleated oocytes.

Well known methods used for isolating and culturing ES cells can be adapted for use with HS cells. Exemplary procedures are provided by Hogan et al., supra, pp. 254-262, 265-272. For example, Hogan et al. describe the optimal culture media as a 7.2-7.4 pH buffered bicarbonate media, such as Dulbecco's modified Eagle's medium, containing glucose and sodium pyruvate, further supplemented with glutamine (2 mM), nonessential amino acids (0.1 mM), mercaptoethanol (0.1 mM) or monothioglycerol (0.15 mM), gentamycin (50 μg/ml), 15% serum (e.g. fetal bovine serum), and leukemia inhibitory factor (LIF). A mixture of trypsin and EDTA in Ca⁺⁺/Mg⁺⁺-free phosphate buffered saline can be used to detach cells from tissue culture dishes and dissociate them from one another. Stem cells are preferably cultured on a feeder cell layer in medium supplemented with LIF to provide factors that enhance the proliferation and maintain the undifferentiated state of stem cells. Fibroblasts, particularly mouse embryo fibroblasts (MEF) and STO mouse fibroblasts, are the preferred feeder cells. Feeder cells should be mitotically inactivated, by treatment with mitomycin C, or gamma radiation. Additional types of feeder cells may used or a feeder-free system may also be used. Detailed protocols for each step in the preparation and maintenance of viable stem cell cultures are provided by Hogan et al., supra.

Similarly, methods for isolating individual colonies of ES cells and expanding them to a sufficient number to allow isolation and screening of DNA can be applied to HS cells. For example, Hogan et al., supra, at pp. 279-281, describe stem cell isolation techniques including: the extraction and transfer of a trypsinized colony to single wells of a culture dish using an automatic pipettor and extraction and transfer of a non-trypsizinized colony to a microdrop of trypsin/EDTA using a sterile, glass Pasteur pipette attached to a tubing and a mouthpiece. Preferred clones can be identified and assayed using rapid techniques and PCR.

To minimize the danger of accumulating chromosomal anomalies, vials of stock should be frozen and stored as soon as possible. Methods for proper freezing and storage of cultures are well known for ES cells, and may be applied to HS cells. See, for example, Hogan, et al., supra, pp.283, setting forth a detailed protocol for freezing, trypsinizing, pelleting and resuspending samples from cell cultures. Methods for differentiation of pluripotent cells are discussed below. These methods are designed to be an illustrative not an exhaustive, list of methods for differentiating pluripotent cells including the HS cells of the present invention. The present invention can be practiced using differentiation methods known in the art, including techniques not recited here, or not yet discovered.

For example, Hole (Cells Tissues Organs, 165:181-189, 1999, and incorporated by reference herein) describes methods for directing the differentiation of hematopoietic cells from embryonic stem cells in vitro that may be adapted to differentiate HS cells. Furthermore, Doetschman et al., Embryol. Exp. Morphol., 87:27-45, 1985, incorporated by reference herein, suggests that the withdrawal of leukemia inhibitory factor (LIF) from ES cells grown in suspended culture results in the formation of cystic embryoid bodies containing blood islands made up of erythrocytes and macrophages. The production of other hematopoietic cells, including neutrophils, mast cells, macrophages and erythroid cells, from stem cells has also been described (See, e.g., Wiles and Keller, Devel., 111:259-267,1991; Keller et al, Mol. Cell. Biol., 13:473-486, 1993a; and Lieschke and Dunn, Exp. Hematol., 23:328-334, 1995; each of which are hereby incorporated by reference herein in their entirety). These methods may be adapted for use with HS cells of the present invention without undue experimentation.

The techniques described by Cho et al., Proc. Natl. Acad. Sci. USA, 96:9797-9802, 1999, incorporated by reference herein, for efficiently differentiating ES cells into mature Ig-secreting B lymphocytes can also be adapted for use with the HS cells of the present invention. Likewise, Dani, Cells Tissues Organs, 165: 173-180, 1999 incorporated by reference herein, describes a method for differentiating ES cells into adipocytes. For example, the treatment of embryoid bodies at an early stage of their differentiation with retinoic acid (RA) for a short period of time appears to be linked to adipogenesis.

Techniques for eliciting the differentiation of stem cells into a variety of neuronal cells and neurons are described by Okabe et al., Mech. Dev., 59: 89-102, 1996, incorporated by reference herein. Likewise, stem cell-derived oligodendrocytes and neurons having particular use in treating injured spinal cords are described by McDonald et al., Nature Medicine, 5:1410-1412,1999 incorporated by reference herein. These techniques can be adapted for use with HS cells of the present invention without undue experimentation.

The use of accessory cell lines, such as OP9, to derive particular cell lineages is also contemplated. See, for example, Nakano el al., Science 265:1098-1101, 1994, (incorporated by reference herein) and Nakayama el al., Blood, 91:2283-2295, 1998 (incorporated by reference herein) that relate to erythroid, myeloid and lymphoid lineages.

Techniques for eliciting the differentiation of HS cells of the invention into follicular cells, as well as epidermal cells are also contemplated. See, for example, Taylor et al., Cell 102: 451-361, 2000 (incorporated by reference herein). The expression of particular regulatory genes may also be used to direct differentiation. See, for example, Hole et al., Blood, 90:1266-1276 1996a, and Battieres Clin. Hematol., 3:467-483, 1997 (incorporated by reference herein), relating to hematopoietic genes. Likewise, nuclear regulatory factors involved in lipid metabolism, including but not limited to PPARs (PPARd and PPAR?) and C/EBPd (C/EBPβ, C/EBPd and C/EBPa), may also be triggers of terminal differentiation of preadipocytes into adipocytes. Such factors would find utility in the context of the differentiation methods of the present invention.

Depending on the function needed, differentiation may be assessed by detecting expression of a gene specific for differentiation, by detecting tissue-specific antigens, by examining cell or tissue morphology, by detecting functional expression such as ion channel function; or by any means suitable for detecting the differentiation of HS cells.

Methods for inducing differentiation of embryonal carcinoma (EC) cells into a variety of embryonic and extraembryonic cell types can be used to induce differentiation of HS cells (Andrews, APMIS, 106:158-168 1998). HS cells can undergo directed differentiation in vitro by exposure to various factors known to trigger cell commitment and differentiation into a desired cell type or tissue. Many in vitro differentiation schemes involve the removal of growth factors known to favor initiation of differentiation. Once these factors are removed from the medium, the stem cells, growing in suspension without feeder cells, form clusters, known as embryoid bodies, within which descendants of all three embryonic germ layers can be found. The presence of certain cell lineages within the embryoid body can be enhanced through supplementation of the medium with additional growth factors and chemicals. The resulting cell population will then contain an increased proportion of a desired cell type, which then can be selectively isolated. See Edwards et al., Modern Trend., 74(1): 1-7, 2000 (incorporated by reference herein) for a discussion of pluripotent stem cells and their use in medicine.

Illustrative examples of such differentiation control factors include but are not limited to cytokines, hormones, and cell-regulating factors such as LIF, GM-CSF, SCF, IL-3, thyroid hormone (T3), stem cell factor (SCF), fibroblast growth factor (FGF-2), platelet derived growth factor (PDGF), ciliary neurotrophic factor. While stimulating cytokines such as GM-CSF, SCF, and IL-3 have been shown to promote differentiation (see Keil F, et al., Ann. Hematol., 79(5):243-8, 2000 (incorporated by reference herein)), inhibitory factors such as LIF has been shown to maintain mouse embryonic stem (ES) cells in the undifferentiated pluripotent state (Zandstra P W, et al., Blood, 96(4):1215-22, 2000 incorporated by reference herein). While SCF has been shown to stimulate the differentiation of chicken osteoclasts from their putative progenitors (van't Hof R J, et al., FASEB J., 1997 Mar;11(4):287-93, 1997 incorporated by reference herein), FGF-2 has been shown to play a role both in initiating lactotrope differentiation and maintaining Prolactin expression in immortalized GHFT cells, thereby suggesting a mechanism for controlling differentiation of stem cells into different anterior pituitary cells (Lopez-Fernandez J, et al., J. Biol. Chem., 275(28):21653-60, 2000 incorporated by reference herein). Platelet-derived growth factor (PDGF-M, -AB, and -BB) supports neuronal differentiation while ciliary neurotrophic factor and thyroid hormone T3 generate clones of astrocytes and oligodendrocytes (Johe K K, et al., Genes Dev., 10(24):3129-40, 1996 incorporated by reference herein).

Differentiation of pluripotent HS cells into various endodermal cell types has great therapeutic implications, including use for transplantation purposes, or to enhance the HS cells can be uptake and processing of nutrients, or to direct pattern formation, induced to differentiate into endodermal progenitor cells by treatment with high doses of RA or by members of the transforming growth factor β superfamily including bone morphogenetic protein (BMP)-2 (Pera and Herzfeld, Reprod. Fert. Dev., 80:551-555 1998). Some HS cell lines can also be induced to differentiate in distinct culture systems. For example, non-neural differentiation can be induced by hexamethylene bisacetamide (HMBA) (Andrews, APMIS, 106:158-168 1998). BMP-2 can be used to specifically trigger differentiation into parietal, or visceral endoderm (Rogers et at., Mol. Bio. Cell., 3:189-196 1992). BMPs are molecules that can induce cartilage and bone growth in vivo, but BMP messages are also expressed in many non-bony tissues, including developing heart, hair follicles and central nervous system, indicating a pivotal role in cell commitment and differentiation.

HS cells can be induced to differentiate by transplantation in vivo, preferably in situ, where the cells undergo histologic and functional differentiation and form appropriate connections with host cells. Endogenous regulation factors located in the transplant site can direct the differentiation of the stem cell into a particular type of differentiated cell or tissue. Alternatively, groups of divergent differentiated cells and/or tissues result from stem cells transplanted to the hypodermis, the peritoneum, and the renal capsule. See Hogan, supra, pp.183 to 184, for a detailed description of the kidney capsule implantation procedure. This technique involves transplanting totipotent or unipotent teratoma stem cells (produced using techniques noted above) into a recipient nude mouse to grow teratoma stem cells under the influence of endogenous factors.

A nude recipient mouse is anesthetized intraperitoneally with 0.015-0.017 ml of 2.5% avertin per gram of body weight. To expose the capsule, a 1-cm long incision is made in the abdomen of the recipient mouse, after wiping the back with 70% ethanol. The abdominal incision is slid to one side, and another incision is then made directly above the level of the ovary to cut the body wall. Blunt fine forceps are then used to pull out the kidney by its fat pad, to be immobilized. The kidney surface is allowed to dry, and using watchmaker's forceps, a small tear is made in the capsule membrane. The membrane is moistened with sterile saline or PBS. A pocket is then made underneath the capsule, and totipotent stem cell populations can then be inserted into the pocket to differentiate in vivo.

A comparable technique to test whether adult or embryonic tissue grows in vivo without rejection, and to obtain teratocarcinomas from early embryos is described in detail in Hogan et al., Manipulating the Mouse Embryo, p.183-184, and is incorporated by reference herein.

Other procedures may be used to cause differentiation of isolated HS cells of the present invention in vivo, such as abdominal, and/or subcutaneous implantation of isolated HS cells. Such procedures comprise using tools and steps that are analogous to those used in implantation under the kidney capsule.

Furthermore, complex organ-like systems can be derived from homozygous stem cells differentiated in vivo using three-dimensional culture conditions. An illustrative system that can be utilized in combination with the present materials and methods is described by Cooper et al. (Biomaterials, March 1991;12(2):243-8, incorporated by reference herein). Cooper describes a three-dimensional culture system utilizing a biodegradable substrate comprised of polyglycolic acid and/or polyglactin mesh. The Cooper system can be adapted for use with HS cell populations to provide three-dimensional models of development and differentiation into various mature tissues. Such three-dimensional cultures not only represent a useful tool in for study of development and differentiation processes but also represent a useful tool in creation of tissues and organs suitable for transplant.

For example, to study the development of epidermal and dermal tissue from HS cells, two biodegradable meshes, polyglycolic acid (PGA), and polyglactin-910 (PGL), are used as carriers for pluripotent HS cells that further differentiate into mature skin tissue when surgically implanted onto the dermis of a recipient mouse. The living tissue replacement vascularizes on a wound, and allows epitheliarization across its surface. Endogenous factors, and the ability of a PGA or PGL graft to promote and support epithelial migration, drive the differentiation of the HS cells.

Isolated HS cells, produced by techniques described in the foregoing sections, are incubated at 37° C. at saturated humidity and 5% carbon dioxide. HS cell cultures are then passaged in a standard fashion, when cells are about 80% confluent. HS cells placed onto meshes are between the sixth and fourteenth passages (approximately 12-42 doublings).

Biodegradable meshes composed of PGA (Davis and Geck, Inc., Danbury, Conn., USA) or PGL (Vicryl, Ethicon, Inc., Somerville, N.J., USA) are obtained in sterile packages from their respective manufacturers. Meshes of individual fibers are either woven or knitted to form a three-dimensional pattern of varying pore size and thickness. Meshes are cut in a laminar flow hood to the dimensions of 2×2 cm. The PGA or PGL grafts are then seeded with 400,000 pluripotent HS cell/graft in a minimal volume of DMEM, and kept at 37° C. and 5% carbon dioxide. After 3-5 hours, the HS cells attach well to the PGL or PGA meshes, and at this time 5 ml of DMEM is added. After 24 hours post-seeding 20 ml of DMEM is added to each culture dish. The PGA/PGL-HS cell cultures are maintained in an incubator for 2-3 weeks until confluence is achieved (sampling is permitted at this time to extract genomic DNA for future investigation of genes activated in the differentiation of HS cells into mature skin tissue.) Inverted phase microscopy is used to confirm that HS cells cover all the mesh pores. The medium is replaced by DMEM and 10% FBS three times a week until grafting procedures are performed.

Athymic mice (Balb/c-nu/nu; Simosen labs, Gilroy, Calif., USA) are kept in isolation rooms prior to grafting. After standard anesthetization, the left-dorsolateral surface of the mouse is sterilized, and a 2×2 cm full thickness wound is created sparing the panniculus carnosis. This results in a 20% total body surface area skin loss. The HS-cell-PGA/PGL mesh is then grafted into the wound and sutured to the mouse skin edges. The mice are given 3 mgs of ceftazidime intraperitoneally for seven days post-surgery. Further, the mice are examined daily for the integrity of the graft, and sacrificed to sample differentiated HS cell. For additional details see Cooper et. al, supra.

As noted above, isolated HS can be differentiated using in vivo and in vitro techniques. An application of the mesh technology, described in the foregoing paragraphs, involves the creation of a three-dimensional model in vitro to study the differentiation of HS cells. The three-dimensional system provides vascularization, and a micro-environment, comprised of endogenous factors produced by the differentiating cells, to mimic an in vivo differentiation system.

Poznansky et al., Nature Biotech., V.18, (July, 2000), describe a technique that assesses optimum conditions for T lymphopoiesis using thymic stromas on matrices of varying dimensions and pore densities, and is incorporated by reference herein. The techniques of Poznansky can be adapted for use with HS cell systems, to create three-dimensional models of cellular development and differentiation.

Isolated HS cells are produced and cultured using techniques described in the foregoing sections. HS cells are then seeded onto tantalum-coated carbon matrices (such as cell foam) of five different dimensions (5×1 mm, 5×2 mm, 10×1 mm, 10×2 mm, and 30×2 mm), and three different pore densities, and then cultured in 24-well tissue culture plates. A suitable culture medium is then added. When the cultures are 80% confluent, as determined by phase-contrast light microscopy, they are transferred to fresh culture plates, cultured for an addition 24 hours. Progenitor cells for the desired tissue, cell type, or group of cells being studied, are then added to the HS-cell culture. For example, to generate mature T-cells, human bone marrow-derived AC133 or CD34 progenitor cells may be used. Progenitor cells are added to the HS cell cultures at cell densities of 100,000, 10,000, and 1,000 cells per well. Nonadherent cells are aspirated at 4, 7, 10, 14, and 21 days, and are assessed and counted by flow-cytomethry flourochrome-congugated antibodies.

Mature cells, such as T-cells, are generated within 14 days because of a xenogeneic environment created by the TS cell-Progenitor cell/Cell foam culture. In the case of thymic development, using bone marrow-derived progenitor cells, a thymic microenvironment is created which consists of stromal cells, cytokines generated by these stromal cells, and three-dimensional vascularization.

### D. SAMPLING HOMOZYGOUS STEM CELLS AT VARIOUS STAGES OF DIRECTED DIFFERENTIATION

The present invention provides methods for detecting genes that are activated, and polypetides that are expressed at various stages of directed differentiation of isolated homozygous (or teratoma) stem cells into the desired types of cells, groups of cells, or tissues.

### i. Sampling by Tissue Microdissection

Tissue microdissection techniques are described by Zhuang et al. (Am J. Pathol., 146:620-625, 1995, incorporated by reference) and Emmert-Buck et al. (Science, 274:998-1001, 1996, incorporated by reference). Tissue microdissection is a useful tool in the procurement of specific cell types from a population of isolated HS cells at various stages of differentiation. Using this technique a pure population of HS cells can be analyzed without interference from neighboring cells. Manual microdissection is performed under an inverted microscope (200× final magnification) on histology slides. 5-15 micron thick sections of the differentiating teratoma system are placed on non-coated glass slides.

Paraffin-embedded tissue is re-cut and stored below room temperature. Deparafinnization is performed immediately before the dissection, by soaking in xylene (×2; five minutes), 95%, 80%, and then 50% ethanol (×2; two minutes/soak), dH₂O (×2; two minutes), and finally 3% glycerol prepared in dH₂O (five minutes). After removing the layer of glycerol/water from the slide, the cells are microdissected by placing a sterile 30 gauge needle on a pencil sized syringe, and then scraping the cell population of interest while viewing the procedure under the microscope. Following microdissection, the tip of the needle is placed into a small microtube with at least 10 μl solution. Shaking the tube detaches the tissue sample from the needle, and subsequently injecting an air bubble suspends the tissue into the solution, and removes any remaining fragments.

For frozen tissue, the procedure for microdissection remains the same, except cut sections (frozen immediately at −70° C.) are allowed to warm at room temperature for approximately 1 minute.

For microdissections resulting in a large number of cells (>100,000) any technique known in the art may be used to extract DNA or RNA from the sample. However, if a small number of cells a procured (800-2000), then the following technique is used to extract DNA for PCR analysis. The issue is resuspended in a 20 ul solution containing 10,M Tris-HCl, 1 mM ethylenediamine tetraacetic acid (EDTA), 1% tween 20, 0.1 mg/ml proteinase K, pH 8.0, and incubated overnight at 37° C. The mixture is then boiled for 8minutes to inactivate the proteinase K, and 0.2-1% of this solution is used in a PCR analysis.

Other techniques for sampling include use of immunoflourescence or histological markers that are known in the art.

### ii. Periodic Cell Sampling and Recovery of Genetic Material

Samples of differentiating HS cells are periodically harvested by the techniques discussed above or other equivalent techniques. The sampling period is hours, days, or weeks depending on the individual tissue undergoing differentiation. RNA and proteins are extracted from microdissected tissue using conventional procedures.

RNA is recovered from sampled cells using conventional methods. For example, total RNA can be isolated from these cells as described in Chirgwin et al., Biochemistry (1970) 18:5294-5299, and Hogan et al., supra at 328-330, both of which are incorporated by reference herein.

### E. INDEX AND ANALYSIS

### i. Librarying Genes and Proteins Expressed by Homozygous Stem Cell Systems at Various Stages of Development

The present invention provides a model for the understanding and discovery of genes that are activated, and/or polypetides that are expressed at various stages of differentiation, particularly over the course of development from isolated homozygous (or teratoma) stem cells to the desired types of cells, groups of cells, or tissues.

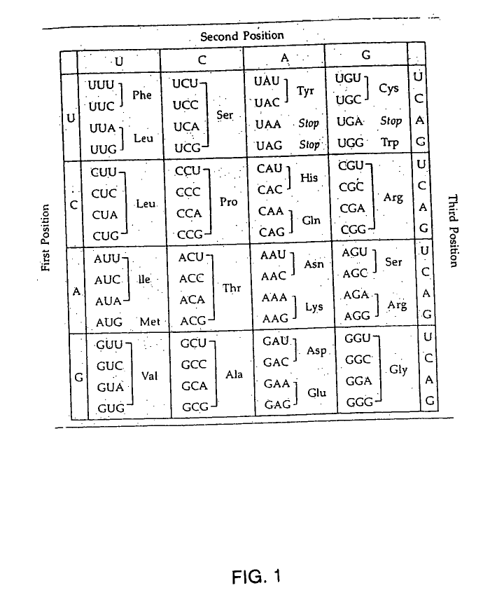

The present invention contemplates each possible variation of a polynucleotide that could be made by selecting combinations based on the possible codon choices listed in Table of FIG. 1 (List of Codons). Further, all such variations are considered to be specifically disclosed and equivalent to the nucleotide sequences provided by the present application. Codons are preferably selected to fit the host cell in which the enzyme is produced. Selection of codons to maximize expression of proteins in a heterologous host is a known technique.

Other DNA molecules that code for polypeptides provided by the present application can readily be determined from the list of codons in the Table of FIG. 1, therefore are likewise contemplated as being equivalent to the nucleotide sequences identified. In fact, as there is a fixed relationship between DNA codons and amino acids in a peptide, any discussion in this application of a replacement or other change in a peptide is equally applicable to the corresponding nucleotide sequence or to the DNA or RNA molecule, recombinant vector, or transformed microorganism in which the sequence is located (and vice versa).

In addition to the specific nucleotides sequences provided in the present application, DNA (or corresponding RNA) molecules of the invention can have additional nucleotides preceding or following those that are specifically listed. For example, poly A can be added to the 3′-terminal; a short (e.g., fewer than 20 nucleotides) sequence can be added to either terminal to provide a terminal sequence corresponding to a restriction endonuclease site; stop codons can follow the peptide sequence to terminate translation; and, the like. Additionally, DNA molecules containing a promoter region or other control region upstream from the gene can be produced. All DNA molecules containing the sequences of the invention will be useful for at least one purpose. All DNA, or RNA molecules are fragmented minimally to produce oligonucleotide probes that are used in the isolation or detection of DNA from biological sources.

Once the nucleotide sequences, encoding proteins at various stages of directed differentiation of HS cells, or the responsible genes are identified, it is possible to produce a gene entirely by synthetic chemistry, after which the gene may be inserted into any of the many available DNA or RNA vectors using known techniques of recombinant DNA technology. Thus, the present invention is carried out using reagents, plasmids, and microorganisms that are freely available and in the public domain at the time of filing of this patent application without requiring a deposit of genetic material.

For example, nucleotide sequences greater than 100 bases long can be readily synthesized on an Applied Biosystems Model 380A DNA Synthesizer as evidenced by commercial advertising of the same (e.g., Genetic Engineering News, November/December 1984, p. 3). Such oligonucleotides can readily be spliced using, among others, the technique of preparing overlapping complementary sequences (e.g, 1-100 of coding strand, 0-50 and 51-150 of complementary strand, 101-200 of coding strand, etc.), followed by hybridizing and ligating the strands. Such techniques are well known and are described in detail in, for example, Davis et al., Basic Methods in Molecular Biology, Elsevier Science Publ. Co., Inc., New York (1986), incorporated by reference herein. Peptides are then expressed in a host organism as described herein.

Automated equipment is also available that makes direct synthesis of many of the polypeptides provided by the present application readily available. Such equipment provides ready access to the peptides of the invention, either by direct synthesis or by synthesis of a series of fragments that can be coupled using other known techniques.

In addition to the specific peptide, polypeptides, and/or proteins provided by this application, peptide fragments based on these sequences, fragments and/or full length amino acid sequences that represent minor variations thereof, are contemplated by the present invention. Such molecules having at least some biological activity similar to the specific peptides, polypeptide, and/or proteins of the present application, find utility in appropriate circumstances, such as, fragments of the present polypeptide sequences may be screened for use in substrate binding site models. Peptide synthesizers can be used to prepare small polypeptide fragments (e.g., less than 100 amino acids) or known techniques of genetic engineering can be used to prepare larger fragments.

The ability to prepare and select peptide fragments having appropriate binding affinity from a larger protein is well known in the art and is described in a number of publications, including patents. See, for example, U.S. Pat. No. 4,629,783, incorporated by reference herein, which describes the preparation of immunologically active fragments of viral proteins that bind with the same antibodies as the entire viral protein.

In addition, minor variations of polypeptide and nucleotide sequences are contemplated as being equivalent to those polypeptide and nucleotide sequences that are described in the present application. For example, it is reasonable to expect that an isolated replacement of a leucine with an isoleucine or valine, an aspartate with a glutamate, a threonine with a serine, or a similar replacement of an amino acid with a structurally related amino acid (i.e., a conservative replacement) will not have a significant effect on the biological activity of the resulting molecule, especially if the replacement does not involve an amino acid at a binding site or other site of biologic activity.

The influence of stimulants, factors, or cytokines on the activity or production of peptides, polypeptide, and/or proteins is readily ascertainable by direct analysis for function. Such an assay relies on ability of a protein/enzyme (or fragment) produced under the influence of stimulants, factors, or cytokines, to carry out the normal function of the natural protein/enzyme (or fragment) using techniques known in the art.

Although genes and corresponding proteins can be prepared by totally synthetic techniques, as discussed above, in preferred embodiments of the invention genetic information is obtained from natural sources, and identified as described herein. The genetic material is first obtained in the form of a gene library, using any of numerous existing techniques. The first of these is to randomly shear genomic DNA, and insert this sheared material into expression vectors. If enough recombinants are generated, there is a good probability of having at least one recombinant in the population that expresses a fusion protein corresponding to the enzyme of interest.

Another strategy for preparing gene libraries is to make complementary DNA (cDNA) copies of the total mRNA population of a differentiating TS cell, and to clone these as recombinant molecules in expression vectors. Use of a cDNA library to obtain genes and gene products involved in cell development is preferred. Such a library is generated in the present application, and screened for expression of gene products.

cDNAs can also be synthesized using methods described in the following references which are incorporated by reference herein: Okayama, et al., Moll. Cell. Biol., 2:161-170, (1982); Gubler et al., Gene, 25:263-269, (1983); Weng et al., Mol. Reprod. Dev., 1:223-224 (1989); and, Rothstein et al., Genes Devel., 6:1190-1201 (1992).

Other forms of cDNA libraries are known in the art, and are particularly described in U.S. Pat. No. 5,846,721 (Soares et al., 1998), and U.S. Pat. No. 6,136,537 (Macevicz, 2000), incorporated by reference herein.

Once sequences of various genes expressed during cell development have been determined, it is no longer necessary to go through the foregoing steps to obtain the genetic material of the present invention. The polymerase chain reaction (PCR) technique can be used to isolate genes from natural sources in a simpler and more direct manner. The PCR technique, including use in diagnosis, is disclosed in U.S. Pat. No. 4,683,202, incorporated by reference herein.

Moreover, microarrays as described by J. Marx (Science, v.289: p.1670-1672 (2000), incorporated by reference herein) can be used to identify and quantify gene expression. Briefly, a slide or chip is systematically dotted with DNA from thousands of genes that can serve as probes for detecting genes that are active in different cells. Oligonucleotide probes developed using the techniques described in the following paragraphs, can be used in conjunction with this technique.

Additionally, other known methods that provide the same results may be used in the preparation of recombinant DNA vectors of this invention. When used in combination with the knowledge of those skilled in the field of genetic engineering, and the techniques disclosed in the foregoing paragraphs, the present application readily enables the isolation of desired genes. Moreover, such isolated genes are useful in therapeutically, or preventatively treating diseases related to cellular development and differentiation, genetic diseases, and cancer.

Expression of amino acid sequences of the present invention can be enhanced by including multiple copies of the responsible gene in a transformed host. Enhancement may be achieved by (a) selecting a vector known to reproduce in a host, (such as pUC8; ptacl2; pIN-III-ompA1, 2, or 3; POTS; pAS1; or pKK2233), (b) inserting exogenous DNA (with an appropriate promotor) that encodes a particular protein into the vector, and (c) transforming host cells (that are preferably enucleated) with the vector containing the insert. Large quantities of protein may then be collected from the transformed host cells that express the exogenous DNA. Other techniques known in the art, or other known means of enhancing peptide expression are also contemplated and incorporated by reference herein.

One common variation known in the art is the preparation of a polypeptide in the form of a fused polypeptide. Such peptides are typically prepared by first identifying a promoter region of a gene known to encode a particular protein, and be expressed in a host. A nucleotide sequence (that encodes a desired protein, or a large portion of the desired protein) may then be inserted at an appropriate position between the promotor and the gene, such that activation of the promotor results in both the known gene and the inserted nucleotide sequence to be expressed. Examples of such fused proteins include beta-galactosidase fused proteins. Moreover, if so desired, said fused polypeptide may be designed such that a specific restriction endonuclease site is present at the junction between the two fused proteins. A restriction endonuclease may then be used to cleave the fused polypeptide such that the desired protein is available in pure form.

In all cases, a particular polypeptide sequence will be encoded when the DNA sequence is functionally inserted into a vector. By “functionally inserted” is meant in proper reading frame and orientation, as is well understood by those skilled in the art. Typically, a gene will be inserted downstream from a promoter and will be followed by a stop codon, although production as a hybrid protein (possibly followed by cleavage) may be used, if desired.

In addition to the above general procedures that can be used for preparing recombinant DNA molecules, and transformed unicellular organisms in accordance with the practices of this invention, other known techniques and modifications may be used in carrying out the practice of this invention. In particular, many recently issued U.S. Patents describe plasmids, genetically engineering microorganisms, and methods of conducting genetic engineering that may be used in the practice of the present invention. These include,

U.S. Pat. No. 4,273,875, incorporated by reference herein, describes a plasmid and a process of isolating the same.

U.S. Pat. No. 4,304,863, incorporated by reference herein, describes a process for producing bacteria by genetic engineering in which a hybrid plasmid is constructed and used to transform a bacterial host.

U.S. Pat. No. 4,419,450, incorporated by reference herein, describes a plasmid useful as a cloning vehicle in recombinant DNA work.

U.S. Pat. No. 4,362,867, incorporated by reference herein, describes recombinant cDNA construction methods and hybrid nucleotides produced thereby which are useful in cloning processes.

U.S. Pat. No. 4,403,036, incorporated by reference herein, describes genetic reagents for generating plasmids containing multiple copies of DNA segments.

U.S. Pat. No. 4,363,877, incorporated by reference herein, discloses recombinant DNA transfer vectors.

U.S. Pat. No. 4,356,270, incorporated by reference herein, describes a recombinant DNA cloning vehicle and is a particularly useful disclosure for those with limited experience in the area of genetic engineering since it defines many of the terms used in genetic engineering and the basic processes used therein.

U.S. Pat. No. 4,336,336, incorporated by reference herein, describes a fused gene and a method of making the same.

U.S. Pat. No. 4,349,629, incorporated by reference herein, describes plasmid vectors and the production and use thereof.

U.S. Pat. No. 4,332,901, incorporated by reference herein, describes a cloning vector useful in recombinant DNA.

Those skilled in recombinant techniques will recognize that the procedures and techniques described above can readily be adapted, without undue experimentation, for use in combination with the present invention.

Particularly contemplated is the isolation of genes using oligonucleotide probes. Such probes are considerably shorter than the entire sequence, but preferably are at least 10-14 nucleotides in length. Intermediate oligonucleotides, ranging from 20 to 500 nucleotides (especially 30 to 200 nucleotides) in length, provide particularly specific and rapid-acting probes. Longer oligonucleotides are also useful up to the full length of the gene. Additionally, both RNA and DNA probes are contemplated.

In use, probes are typically labeled in a detectable manner (e.g., with ³²P, ³H, biotin, or avidin), and are incubated with single-stranded DNA or RNA extracted from the organism in which a gene is being sought.

Although probes are normally used with a detectable label that allows easy identification, unlabeled oligonucleotides are also useful, both as precursors of labeled probes and for use in methods that provide for direct detection of double-stranded DNA (or DNA/RNA heteromers). Accordingly, the term “oligonucleotide probe” refers to both labeled and unlabeled forms.

### ii. Cataloguing the Library Information into a Searchable Database

The gene and protein information obtained from the nucleic acid samples, as described in the above section, is stored as a centralized collection of data on the Internet or World Wide Web and accessible via a personal computer. In an alternate embodiment, information can be stored in a Local Access Network (“LAN”) database that receives updates. The information is stored at a remote server. The remote server includes a database management system (DBMS), which collects and stores all information that is accepted in a database system. The server database management system allows for access to the information within the database and processing thereof.

The end user is able to build and submit customized queries to retrieve genetic information. Using a browser, the end user is able to submit their queries to the system. When data is entered into the system, the data is given an identification number. This identification number may be used as a key field. The end user may also enter as a query the technical name of the gene or protein. Further, the database may also contain various searchable fields. For example, a user may customize a query using key fields for nucleotide sequences, amino acid sequences, sequence length and/or molecular weight, gene and/or protein names, definitions (assigned by conventional methods), source, organisms, keywords, and/or dates of identification and/or submission. Advanced searches may also be performed by customized queries using additional key fields such as, (a) location (e.g., a nuclear protein, a cell wall antigen), (b) cellular source (e.g., produced by neural cells, epithelial cell, or a hematopoietic precursor) and/or (c) biological characteristics (such as specific biological properties of proteins found in nature, for example, specific binding, catalyzing and/or inhibitory properties).

In another embodiment, the electronic database may contain non-searchable fields that are purely descriptive, providing general information regarding the gene or protein. For example, a field with the history of a gene and/or protein, usage, or any other comments regarding a gene or protein may be added to the database.

Once an end user has submitted a request, the DBSM will automatically retrieve the requested gene and/or protein, as well as similar or related genes and/or proteins (such as other proteins belonging to the same family as the requested protein). The system is designed to identify all similar genes or proteins within a specified error tolerance. Alternatively, the system allows users to input specific error tolerance. For example, the user may specify the query generate hits that have a 75% or greater homology, or specify a maximum number of hits to be generated that are automatically listed in decreasing rates of homology. Additionally, query results are listed with the exact match at the top of the results and the least similar match at the bottom by default.

A tolerance band allows users to find not only a protein or gene that is identical to the one inputted, but also other proteins that have high degrees of homology, and/or new members of an established family. Proteins that are substantially similar (80% homology or greater) are expected to be “related”, that is, having similar properties.

The query results are formatted in a manner such that a complete sequence of the gene or protein depicts in both its character form as well as a pictorial of the sequence. Finally, the results also include a brief description of the gene, where the gene was discovered, and when the gene was first sequenced.

### iii. Using the Library Information to Understand Cellular Development and Disease Pathology

Isolated mRNA, cDNA, and/or protein expression profiles may be generated using the methods disclosed in the present application. Such profiles may be used to identify specific genes involved in each step of normal development of each type of mature tissue. Moreover, many desired genes may only be expressed transiently in these steps. Periodic sampling techniques disclosed by the present invention may be used to identify such genes, such that a comprehensive picture of all genes expressed during cellular differentiation into various tissue types may be developed. Specific applications of the present technology include tissue regeneration, anti-cancer therapy, data on the progression of cancers for therapeutic or prophylactic treatment, gene therapy or gene manipulation, and data on developmental anomalies for therapeutic or prophylactic intervention. For example, in the treatment of genetic or developmental diseases, the methods provided by the present invention are used to identify ‘targets’ (i.e. critical gene mutations that occur during tissue differentiation), such that comprehensive intervention strategies such as genetic manipulation using teratoma stem cells, and/or targeted drug treatments, may be developed.

The following examples are provided for purposes of illustration only and are not to be considered limiting of the invention.

### F. EXAMPLES

### Example 1

Homozygous Stem Cell Formation, and their Differentiation Into Progenitor Cells and Various Tissues of the Three Embryonic Germ Layers Within Stemplasms

### Example 1(a)

Derivation of HS Cells from Mouse Post-Meiosis I Oocvtes By Activation Followed by Prevention of the Extrusion of the Secondary Polar Body

Three eight-week old female C57/BDA2 F1 mice (Charles River Laboratories, Wilmington, Mass.), were superovulated by sub-peritoneal injections of 5 IU/100 ul of pregnant mare's serum gonadotropin (PMS; PCCA, Houston, Tex. (29-1000-1BX)), and 5 IU/100 μl of human chorionic gonadotropin (HCG; Sigma, St. Louis, Mo., (C8554)) with 48 hours apart between the injections. Seventy-three oocytes were harvested about 17 hours after the HCG injection, and the cumulus was removed by incubating the freshly obtained oocytes in a drop (˜300 μl) of hyaluronidase (Sigma, H4272) diluted in M2 media (M7167, Sigma) at final concentration of 0.3 mg/ml, followed by 3 washes with HEPES buffered M2 media before further handling. Oocytes were then activated by treatment with 5 μM calcium ionophore (Sigma, C7522) solution at room temperature for five minutes followed by 2 washes with HEPES buffered M2 media. The oocytes were further subjected to incubation in 5 mM 6-dimethylaminopurine (6-DMAP) (Sigma, D2629) in M16 bicarbonate-buffered culture media (Sigma, M7292) at 5% CO₂ and 37° C. for 3 hours. After the incubation, oocytes were washed three times with M16 media and incubated in a drop of M16 media under mineral oil until blastocyst-like masses developed (4 to 5 days). The blastocyst-like masses then naturally hatch out of the surrounding zona shell, and the blastocyst-like masses were then transferred to a mitomycin-C treated murine embryonic feeder cell layer and cultured for at least 15 days in ES medium (DMEM (Gibco, Life Technologies, Rockville, Md., 11995-065) with 20% fetal bovine serum (Gibco, 16141-079) and 1,400 U/ml of LIF (Chemicon, ESG1106) for stem cell line formation.

Alternatively, immunosurgery was performed to remove trophoblast cells from blastocyst-like masses before culturing on the feeder by the following procedure. Hatched blastocyst-like masses were first incubated with anti-mouse Thy-1 rabbit serum (1:10 dilution in stem cell medium, Accurate Chemical, Westbury, N.Y., ACL2001) and anti-human lymphocytes rabbit serum (1:10 dilution in stem cell medium, Accurate Chemical, CL8010) for one hour at 37° C. After washes three times with M2 medium the blastocyst-like masses were then incubated with guinea pig complement (1:10 dilution in stem cell medium, Accurate Chemical, ACL4051) for 30 minutes at 37° C. The complement-treated cell masses were then washed 3 times in the M2 medium and transferred to a mitomycin-C treated murine embryonic feeder cell layer for further culturing in stem cell medium consisted of 80% Dulbecco's modified Eagle's medium (no pyruvate, high glucose formulation; Gibco-BRL) supplemented with 20% fetal bovine serum (Gibco-BRL), 1 mM glutamine, 0.1 mM -mercaptoethanol (Sigma), 1% nonessential amino acid stock (Gibco-BRL) and 1,400 U/ml LIF (Chemicon, ESG1106).

After 15 days, inner cell mass-derived outgrowths formed and were dissociated into clumps by mechanical dissociation with a micropipette and replated on murine fibroblasts feeder in fresh stem cell medium. Individual colonies with a uniform undifferentiated morphology were individually selected by micropipette, mechanically dissociated into clumps, and replated. Once established and expanded, cultures were passaged by 5 minutes exposure to Trypsin/EDTA solution (0.05%/0.5%, Gibco-BRL) followed by further culturing on feeder cells and fresh stem cell medium.

Murine embryonic fibroblasts feeder cells were purchased from Stemcell, Inc. (00308), and passaged 2-3 times before use. To mitotically inhibit the feeder cells, one 60-mm dish of confluent-expanded feeder cells was treated with 5 ml of 10 μg/ml mitomycin-C (Sigma, M4287) in DMEM/10% FBS medium at 37° C. for three hours. Treated feeder cells were then washed with 5 ml DMEM/10% FBS three times, and collected by trypsinization at 37° C. for 5 minutes, followed by neutralization with 5 ml DMEM/10% FBS medium and centrifugation at 1000 rpm for 5 minutes. The mitomycin-treated cell pellet obtained was then resuspended in 15 ml DMEM/10% FBS medium, plated on three 60-mm dishes (5 ml of cell suspension/dish), and incubated at 37° C. overnight before use.

### Example 1(b)

Derivation of HS Cells Homozygous for a Specific Genotype and an Immunotype From Female C57/DBA2 Hybrid Mice

Five female C57DBA2 hybrid mice heterozygous for most genetic loci and H-2 (mouse MHC) were superovulated as described in example 1(a) for HS cells population. Eleven cell lines were derived and were propagated in culture and then cryopreserved. Cells from each line at around passage 5 were immunotyped for I-E-beta in H-2 loci (sequences of PCR primers are: Eb1:CAG MC CTG AGT CCT GGG CG; Eb2:AGC AGA CCA GGA GGT TGT GG) and genotyped for D2mit42 using microsatellite markers (Research Genetics).

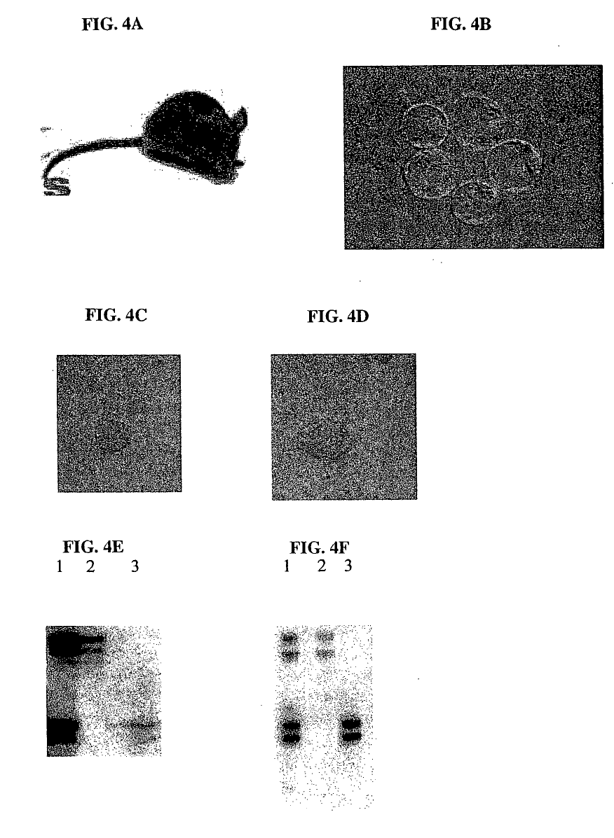

Methods for genomic DNA extraction, PCR amplification, and genetic analysis (using D2mit42 microsatellite marker) of mouse HS cells were as describe in example 1(f). Immunotyping of H-2 (I-E-beta) was performed by separating the PCR products of the H-2 alleles using single-strand conformational polymorphism (SSCP) analysis on a MDE gel (FMC Bioproducts). Results of genotyping and immunotyping of two representative HS cell lines derived from C57/DBA2 hybrid mice are shown in FIGS. 4A-F, in which a C57/DBA2 mouse (FIG. 4A), and her superovulated oocytes-derived blastocyst-like masses (FIG. 4B) and two representative colony (FIGS. 4C and 4D) from two different HS cell lines are shown. The genotype (FIG. 4E) and immunotype (FIG. 4F) of the donor mouse (Lane 1), HS cell line-1 (Lane 2), and HS-cell line-2 (Lane 3) are also demonstrated.

### Example 1(c)

Development of Blastocyst-Like Cell Masses From Human Diploid Oocytes By Activation Followed By the Prevention of the Extrusion of the Secondary Polar Body.

Female ovum donors underwent down-regulation with leurpolide acetate (Lupron: TAP Pharmaceuticals, Deerfield, Ill.) and then began COH (Controlled Ovarian hyper-stimulation) by receiving follicle stimulating hormone (FSH) (Serono, Gona-F) treatment at a dosage of 300 IU/day to induce an appropriate multifollicular response. When ultrasonographic criteria for follicular maturity were met, a single 10,000 IU dose of human chorionic gonadotropin (hCG) (Serono, Profasi) was administered, and transvaginal follicular aspiration was performed approximately 36 hours after hCG administration. Cumulus from retrieved oocytes were removed by exposing them to 80 IU/ml hyaluronidase for approximately 30 seconds followed by HEPES-buffered human tubal fluid supplemented with 10% humans serum albumin (HEPES-HTF-HSA) (InVitroCare, Inc., San Diego, Calif., 2002 and 2101).

To accomplish mitotic activation, the cumulus free mature M-II oocytes were treated with 5 μM calcium ionophore (A23187, Sigma) in HEPES-HTF-HSA for 5 minutes at 33° C. followed by 3 washes with HEPES-HTF-HSA and incubation in 1 to 5 mM 6-dimethylaminopurine (6-DMAP, Sigma) in IVC-TWO (InVitroCare, 2008) for 3 to 5 hours at 37° C. The activated oocytes were incubated in IVC-ONE medium (InVitroCare, 2006) for 3 days, and further incubated in IVC-THREE (InVitroCare) for 2 days for cell division and blastocyst-like mass formation.

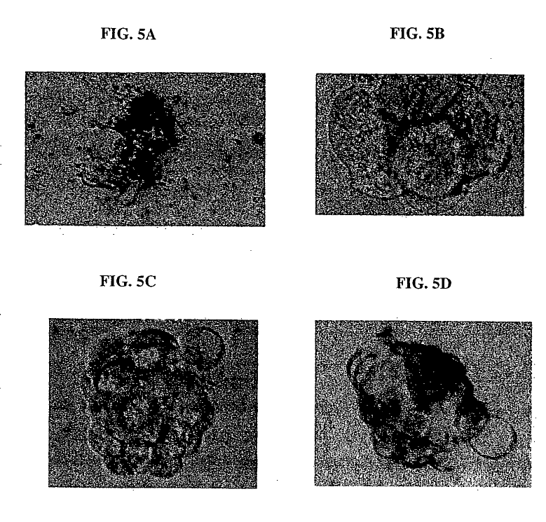

Alternatively, oocytes were co-cultured with STO mouse feeder cells. On day 6 assisted hatching was performed under a micromanipulator under which a blastocyst-like mass was clamped with the holding pipette (syringe suction system) so that the micro-needle filled with acidified tyrodes solution (Medi-Cult, 1060-0002) at the 3 o'clock position is exposed to the empty perivitelline space to expel the acidified tyrodes solution gently over a small (30 microns) area by holding the needle tip very close to the zona. Expulsion of the acidified tyrodes solution was ceased immediately when the inside of the zona is pierced or softened. The blastocyst-like cell mass would then release from the weakened zona. After blastocyst-like mass detached from zona, immunosurgery was performed to remove trophoblast cells by incubating the mass with anti-human Thy-1 (1:10 dilution in IVC-THREE, Accurate Chemical, Westbury, N.Y., CBL415-CD90) and anti-human lymphocytes (1:10 dilution in IVC-THREE, Accurate Chemical, CL8010) for one hour at 37° C. After washes three times with IVC-THREE medium the blastocyst-like mass was then incubated with LOW-TOX guinea pig complement (1:10 dilution in IVC-THREE, Cedarlane, CL4051) for 30 minutes at 37° C. The complement-treated cell masses were then washed 3 times in the IVC-THREE medium and transferred to a mitomycin-C treated STO (ATCC) feeder cell layer and cultured in stem cell medium consisted of 80% Dulbecco's modified Eagle's medium (no pyruvate, high glucose formulation; Gibco-BRL) supplemented with 20% fetal bovine serum (Gibco-BRL), 1 mM glutamine, 0.1 mM -mercaptoethanol (Sigma), 1% nonessential amino acid stock (Gibco-BRL) and 1,400 U/ml LIF (Chemicon, ESG1106). See FIG. 5A.

### Example 1(d)

Development of Blastocyst-Like Cell Masses from Human Post Meiosis I Diploid Oocytes By Activation Followed By Allowing the Extrusion of the Secondary Polar Body and Genomic Self-Replication

Procedures for superovulation, oocyte retrieval, and the subsequent removal of cumulus were as described in Example 1(b). To accomplish mitotic activation, the cumulus free mature M-II oocytes were subjected to sham intracytoplasmic sperm injection (ICSI) to mimic activation introduced by sperm followed by incubation with 25 μM calcium iononphore (A23187, Sigma) for 5 minutes at 33° C. Oocytes activated in this manner extrude the secondary polar body and become haploid. Such haploid oocytes were incubated in IVC-ONE medium (InVitroCare, Inc.) for 3 days, and further incubated in IVC-THREE (InVitroCare) for 2 days for cell division and blastocyst-like cell masses formation. The subsequent manipulations of the blastocyst-like mass were as described in Example 1(b). Haploid oocytes resulting from activation are able to self-replicate their genome without cytokinesis and give rise to diploid cells (Taylor, A. S., et al., Hum. Reprod. 9(12):2389-97 (1994); Kaufman, M. H. et al., J. Embryol. Exp. Morphol. 73:249-61 (1983). See FIGS. 5B-D.

### Example 1(e)

Mouse HS Cell Growth, Differentiation of Such HS Cells Under Mouse Kidney Capsule, And Embryoid Body Formation of Such Cells

HS cells obtained from blastocyst-like masses as described in Example1(a) were seeded on mouse feeder cells in 0.1% gelatin coated dishes (10 cm) with stem cell medium as described in Example 1(a) to colony formation.

One colony of HS cells was dissected into several pieces and implanted in one of the two kidney capsules of 26 hybrid mice to induce stemplasm formation. Stemplasms were then harvested by sacrificing the mice in the post-implantation week 1, 3, 6, 9.5, 10.5, 11, 12, and 14. Half of each stemplasm was fixed in formalin for morphological studies, and the other half was frozen in −80° C. for molecular characterization. Stemplasm started to be formed to a visible size around week three. By staggering the harvesting of stemplasms, various tissue types that developed within the stemplasms were studied. All tissue types identified herein were produced within said stemplasms. Stemplasm genotype was verified by PCR-based allelic analysis.

To create embryoid bodies (EB), HS cells on a 60 mm dish were first washed with PBS twice. 1 ml of Trypsin/EDTA solution was then added, and cells were held at a temperature of 37° C. for five minutes. 5 ml of ES medium was then added, and cells were lifted by a cell scraper and spun down at 1000 rpm for five minutes. The cell pellet thus obtained was then resuspended in 5 ml stem cell medium without LIF, and the cell number was counted. Cells were then seeded onto a suspension dish with lid and vent (Nalge Nunc International, 171099, 35×10 mm) at 2×10⁶/10 cm dish. Cells were fed in stem cell medium for 4 days, where medium was changed every two days by transferring cells into 15 ml tubes, waiting about five minutes until the cells settle to the bottom of the tube, then replacing medium. Cells were then transferred back to the original dish and were allowed to aggregate into embryoid bodies for further differentiation.

### Example 1(f)

Differentiation of Human HS Cells Within Teratomas, and the Genetic Homozygosity of Such Differentiated Tissue.

Thirty-one teratomas were retrieved from the files of the Armed Forces Institute of Pathology, Washington, D.C., and Department of Pathology, New York University. New York, N.Y. (Dr. J. Liu). A variety of different kinds of exclusively differentiated tissue were found in twenty ovarian tumors from female patients. Differentiated tissue was found to be diploid as confirmed by FISH analysis carried out in representative cases using methods known in the art, and alpha-satellite probes to chromosomes 3 and 8. Between 3 and 12 histological areas of undifferentiated and differentiated tissue found in seven ovarian tumors from female patients and four testicular tumors from male patients were identified and selectively microdissected from each case for genetic analysis. In each case, differentiated tissue was found to be genetically homozygous, and undifferentiated tissue was found to be genetically heterozygous.

Microdissection. Unstained 6-micron sections on glass slides were deparaffinized with xylene, rinsed in ethanol from 100% to 80%, briefly stained with hematoxylin and eosin, and rinsed in 10% glycerol in TE buffer. Tissue microdissection was performed under direct light microscopic visualization. From each case, between 6 and 12 areas of different tissue differentiation were separately micro dissected for genetic analysis. In addition, several areas of normal, non-neoplastic tissue were procured.

DNA Extraction. Procured cells were immediately resuspended in 25 μl buffer containing Tris-HCl, pH 8.0; 1.0 mM ethylenediamine tetraacetic acid, pH 8.0; 1% Tween 20; and 0.5 mg/ml proteinase K, and were incubated at 37° C. overnight. The mixture was boiled for 5 minutes to inactivate the proteinase K and 1.5 μl of this solution was used for PCR amplification of the DNA.

Genetic Analysis. In order to reliably identify homozygosity in the limited amounts of DNA that were available after microdissection, multiple different microdissected tissue samples were analyzed with up to 14 distinct highly polymorphic microsatellite markers including DIS1646 and D1S243 (1p), D3S2452 (3p), D5S346 (5q), D7S1822 (7q), Ank-1 (8p), D9S171 (9p), D9S303 (9q), Int-2and PYGM (11q), IFNA (9p), D17S250 (17q), CYP2D (22q), and AR (Xq). Each PCR sample contained 1.5 μl of template DNA as described above, 10 pmol of each primer, 20 nmol each of dATP, dCTP, DGTP, and DTTP, 15 mM MgCl₂, 0.1U Taq DNA polymerase, 0.05 ml [³²P]dCTP (6000 Ci/mmol), and 1 μl of 10× buffer in a total volume of 10 μl. PCR was performed with 35 cycles: denaturing at 95° C. for 1 min, annealing for 1 min (annealing temperature between 55° and 60° C. depending on-the marker) and extending at 72° C. for 60 sec. The final extension was continued for 10 minutes. Labeled amplified DNA was mixed with an equal volume of formamide loading dye (95% formamide, 20 mM EDTA, 0.05% bromophenol blue, and 0.05% xylene cyanol).

Samples can be denatured for 5 min at 95%, loaded onto a gel consisting of 6% acrylamide (acrylamide:bisacrylamide 49:1), and electrophoresed at 1800 V for 90 minutes. After electrophoresis, the gels can be transferred to 3 mm Whatman paper and dried. Autoradiography can be performed with Kodak X-OMAT film (Eastman Kodak, Rochester, N.Y.).

Results. Differentiated teratomous tissue showing consistent homozygosity of the same allele included microdissected samples of squamous epithelium, glia, and cartilage (analyzed with markers Ankl (top) and D1S1646 (bottom)). Normal ovarian tissue was included as control.

In a subset of teratomas, differentiated teratomous tissue found to have discordant homozygous alleles (analyzed with markers Int-2, D9S303, D1S1646, D3S2452, and Ankl) included samples of epidermis, sebaceous gland, respiratory epithelium, and glia. Normal ovarian tissue was included as a control. In such tumors, it is believed that allelic heterozygosity results from the initiation of tumorigenesis before meiosis I in germ cells. After teratogenic tumor cell initiation, random, independent events then lead to progenitor cells with a postmeiotic genotype.

A series of ovarian teratomas and testicular germ cell tumors containing both differentiated and undifferentiated tissue were also analyzed. In each tumor, both undifferentiated and differentiated tissue elements were procured. Homozygous and heterozygous components were detected using markers D3S2452, D3S303, CYP2D, and D17S250. Normal ovarian and testicular tissues were included as controls. Heterozygous alleles were detected in undifferentiated tissue elements including immature squamous epithelium, neural tissue (sometimes from separate areas of neural tissue within the same tumor), cartilage, glandular structures, and mesenchyme. Differentiated tissue elements isolated from the same tumors by microdissection were found to be homozygous for the same markers. Mature elements tested included: sebaceous gland tissue, hair follicle, and mature squamous epithelium (sometimes from separate areas of squamous epithelium within the same tumor). In some tumors, differentiated elements showed opposite homozygous alleles, indicating recombination or suggesting that various elements arose separately from distinct postmeiotic cells.

### Example 1(g)

Derivation of Progenitor Cells from Human HS Cells Primary Differentiation

HS cells grown on 60 mm dish (Falcon, #353802) with primary embryonic fibroblast layer and/or 0.1% gelatin coated dishes are trypsinized with 1.5 ml Trypsin/EDTA (Invitrogen, #25300-050) and transferred to 1.5 ml ES-LIF medium in a 15 ml conical tube. Cells are then spun down at 1200 rpm, and the supernatant is removed. The cell pellet is resuspended into single cell suspension in 2 ml ES-LIF medium, and cultured as suspension cells in suspension culture-35*10 mm-dishes (NalgeNunc, #171099) at a density of 1-3×10⁶ cells to allow stem cells to form rounded spherical clusters, known as embryoid bodies (EBs) for 4-6 days. Forming EBs are washed every two days by transferring the EBs to 15 ml conical tubes, and then allowed to settle to the bottom. The supernatant is removed and new ES-LIF is added. EBs are then transferred back into suspension culture dish. HS cells grown as embryoid bodies are comprised of all the germ cell layers, ectodermal, endodermal, and mesodermal.

Ectodermal Progenitors. After 4-6 days, EBs are trypsinized in 1 ml of Trypsin/EDTA, washed in 4 ml ES-LIF medium, and resuspended into single cell suspension in DMEM/Knockout medium (Invitrogen, #10829-018) supplemented with 10% Serum Replacement (Invitrogen, #10828), and G5 (Invitrogen, #17503), N2 (Invitrogen, #17502-048) or beta NGF (100 ng/ml) (R&D Systems, #256-GF). These cells are cultured at 3-5×10⁵/3 ml in fibronectin-coated 35 mm dishes (50 ug/ml)(Sigma, #F-0895) for 10 days, with media changes every two-three days.

Alternatively, the EBs are cultured in 0.1% gelatin-coated dish in ES-LIF medium for 1-2 days, and then the medium is changed to serum-free medium supplemented with Insulin (5 ug/ml), Selenium chloride (0.015 nM), Transferrin (50 ug/ml), and fibronectin (5 ug/ml)(Sigma) for 6 days. The cells are trypsinized, and single cell suspensions are cultured in N2 medium (serum free-DMEM/F12 supplemented with N2 (Invitrogen, #17502-048), B27 (Invitrogen, #17504-44), and bFGF (10 ng/mL) (Invitrogen, #13256-029)). Cells are then counted and seeded at a density of 2-5×10⁴ cells/well/400 uL N2 medium in 24-well plates pre-coated with poly-L-ornithine (15 ug/ml)(Sigma, #P36550), and expanded for six days.

These progenitors are further differentiated into different neuronal cell types by adding G5, RA, FGF, NGF, GNDF, or BNDF. They are also maintained in their presence conditioned media for cell expansion.

Mesodermal Progenitors. For mesodermal progenitors, the single cell suspension in DMEM/Knockout medium supplemented with 10% Serum Replacement and beta-NGF as described above are cultured for 10 days with media change every two/three days. After this period, the cells are further cultured in Activin A supplemented (20 ng/ml) (Sigma, #A4941) conditioned medium for another 10 days for heart progenitor cells. Alternatively, for kidney and Mullerian duct progenitor cells the cells are further cultured in Activin A supplemented (20 ng/ml) (Sigma, #A4941) conditioned medium for 4-6 days after which 2 ng/ml of TGF-beta (R&D Systems, #) is added to the medium, and the cells are cultured for another 4-6 days.

Endodermal Progenitors. For endodermal progenitors, the single cell suspension in DMEM/Knockout medium supplemented with 10% Serum Replacement, along with G5 or beta-NGF on laminin-coated (10 ug/ml)(Sigma, #L2020), or Collagen I-coated (10 ug/ml)(Sigma, #C-7661) is cultured for 10 days. HGF (20 ng/ml) and/or TGF-alpha (2 ng/ml) are added to the medium to replace G5 or beta-NGF, and the cells are cultured for another 6-8 days.

Alternatively, EBs are plated onto Collagen I-coated dishes and cultured in ES-LIF medium for 4 days. FGF (20 ng/ml) is added and the cells are cultured for another 3 days. After this period, HGF (20 ng/ml) and/or TGF-alpha (2 ng/ml) are added and cultured for another 6 days.

EBs are also transferred to laminin-coated adherent dishes (10 ng/ml) (Sigma, #L2020) or 0.1% gelatin coated 35*10 mm adherent dish, and cultured 1-2 days in ES-LIF medium. The medium is removed and serum-free DMEM/F12 (Invitrogen, #11330-0321) medium supplemented with Insulin (5 ug/ml)(lnvitrogen, #11882), Selenium chloride (0.015 nM)(Sigma, #S5261), Transferrin (50 ug/ml) (Sigma, #T-2036), and Fibronectin (5 ug/ml) (Sigma). This medium is designated as ITSFn medium. Cells are fed for 6 days in ITSFn medium, where medium is changed every two days.

### Example 1(h)

Development and Isolation of Homozygous Progenitor Cells from Transplanted HS Cells

To obtain homozygous progenitor cells, pluripotent HS cells derived from methods disclosed in the foregoing in the foregoing description and examples are transplanted into immuno-compromised mice under kidney capsules and are allowed to grow in vivo for 4 to 6 weeks. The cell mass obtained is then minced into single cells and cultured on feeder cells for further propagation and development into cell lines.

To assess the lineage commitment (the types of progenitor cells), gene expression assays, such as RT-PCR, northern blot, immunohistochemistry, and so forth, are performed for known lineage-specific markers, for example, NF-H, keratin, D-beta-H for the ectoderm, enolase, CMP, rennin, kallikerein, WT1, delta-globin, beta-globin for the mesoderm, and albumin, alpha-1-AT, amylase, PDX-1, insulin, alpha-FP for the endoderm progenitor lineages.

### Example 2

Selection of HS Cells Having a Target Immunotype from Populations of HS cells Derived from Material Donated by a Relative (e.q, Parent of the Intended Recipient)

Oocytes are obtained from the recipient's mother by super-ovulation using methods described in the foregoing examples. Once HS cell populations have developed, following methods described in the foregoing examples, a sample of each HS population can be subjected to in vitro differentiation for the optimal expression of HLA molecule (e.g., hemapoietic lineage), the HS-derived sample cells from each population are then tested for HLA-A, -B, and -C specificities using the microlymphotoxicity assay. The test is performed in a 60- or 72 well microlymphotoxicity plate. A panel of antisera, obtained from a commercial source, are selected and prepoured onto the plates and cooled in a −40° or −70° C. freezer. 0.5-1 μl of HS cell suspension (prepared by suspending HS cells in RPMI, or a desired diluent for typing, to 1.5×10⁶ HS-derived sample cells per milliliter using standard techniques) is dispensed into each well of the plate and incubated at 20-22° C. for thirty minutes. 2-5 μl of complement is then added to each well of the plate and incubated at 20-22° C. for sixty minutes. Complement is available commercially as pooled rabbit serum in a freeze-dried form. 1 μl of 5% Eosin solution followed by 1 μl of formaldehyde is then added to each well. Using an appropriate microscope, cell death in each well is established as determined by the amount of dye fixed onto the cells. A standard scoring system is used, from 0 to 8, where 8 is 80-100% representing a strong positive, and 2 is 20-40% representing a doubtful positive.

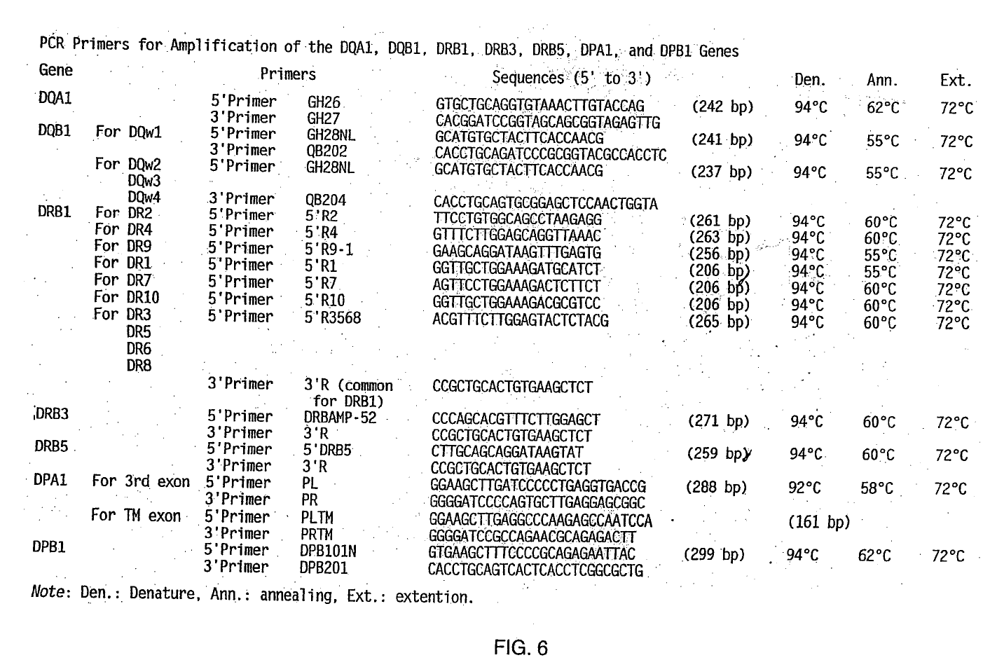

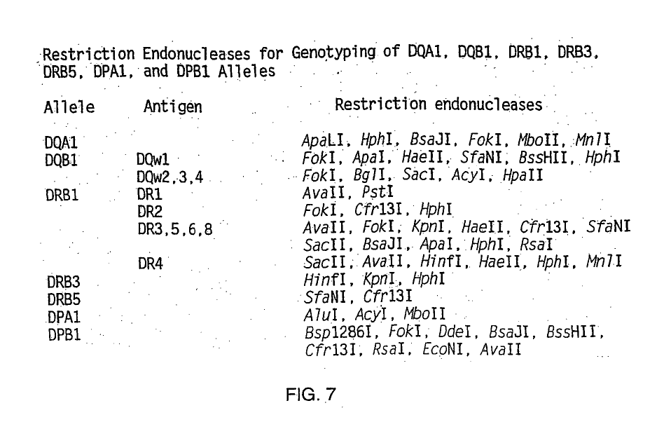

The sample cells are then tested for HLA-DR, -DQ and -DB using PCR-RFLP analysis. DNA is extracted from HS cells using standard procedures known in the art. Extracted DNA, 200-300 ng, is then amplified by PCR with 2.5 U of the Taq polymerase. PCR primers used for amplification of HLA class II are listed in FIG. 6. After amplification, 4-7 μl of a reaction mixture are added to a solution containing a restriction enzyme chosen from the list in FIG. 7, and an appropriate restriction buffer, and incubated for 3 hrs. Twenty-nine different enzymes are used for full class II typing. Samples of the amplified DNAs cleaved by restriction enzymes are then subjected to electrophoresis using a 12% polyacrylamide horizontal gel in a mini-apparatus (e.g. Mupid-2 obtained from E-Y laboratories Inc., San Mateo, Calif.). RFLP fragments are detected by staining with ethidium bromide.

The immunotype of the recipient is determined using the serological and molecular methodology described above. 20 ml of venous blood is obtained from the recipient (target) into sodium citrate anticoagulant (1 ml of 3.3% sodium citrate per 10 ml of blood.) The citrated blood is then diluted with an equal volume of heparinized HBSS (1 ml of 1000 U/ml heparin to 100 ml of HBSS). A 10 ml volume of diluted blood is layered onto 4 ml of Ficoll-isopaque in 17-120 mm screw top centrifuge tubes using a pipette. The tubes are then centrifuged at 700×g for fifteen to twenty minutes. Red blood cell and polymorphonuclear cells form a pellet at the end of the tube, and lymphocytes form a discrete layer at the interface of Ficoll-isopaque. The lymphocyte layer is aspirated into another tube, topped off with HBSS, and centrifuged again at 250×g for five minutes. The lymphocyte pellet is then resuspended, washed with HBSS, and centrifuged again at 120×g for five minutes. This step separates most of the platelets, suspended in the supernatant, while pelleting the lymphocytes at the bottom of the tube. The HBSS is discarded, and the lymphocyte pellet is resuspended in HBSS, and spun for the last time at 250×g for five minutes. The HBSS is then discarded, and the lymphocyte pellet is resuspended in RPMI or a desired diluent for typing. HLA-A, B and C typing procedure described above with the sample stem is repeated to determine the target HLA-haplotype.

Another 20 ml or less of venous blood is obtained from the recipient for molecular typing of HLA class II. Genomic DNA extracted from the blood cell of the recipient using techniques known in the art is amplified using PCR. An RFLP analysis as described above is then performed.

The recipient's HLA haplotype may be homozygous or heterozygous, and in either situation one HLA haplotype (e.g., maternal II) will match with one of the HS cell populations derived from the mother's oocytes. Once a HS population is determined to have the target immunotype, it may then be differentiated in vivo, or in vitro using the techniques described in U.S. patent application Ser. Nos. 09/997,240, and 10/032,495, fully incorporated by reference herein, and transplanted into the recipient.

### Example 3

Selection of HS Cells Having Target Genotype from Populations of HS Cells Derived from Material Extracted from Intended Recipient (Self Donor)

In those instances where the intended recipient of the transplant suffers from a genetic disorder linked to the expression of a mutant gene sequence, it becomes necessary to select the population of HS cells that are homozygous for non-mutant haplotype (e.g., the “target” genotype). An exemplary protocol for the selection for genotype is as follows:

In this example, the intended recipient is a women afflicted with sickle cell anemia. A number of human disease states have been attributed to genetic mutations effecting one or more of the genes encoding hemoglobin polypeptide chains, including sickle cell anemia, which results from a point mutation in the hemoglobin β-chain. The genetic sequence of the mutant gene associated with sickle cell anemia has been disclosed and extensively studied. See, for example, Saiki et al., 1985, Science, 230, 1350-1354, incorporated by reference herein.

A series of populations of HS cells are created from oocytes harvested from the intended recipient according to the procedures described above in Example 1. The populations are then assayed for genotype. Only those populations that homogeneously carry the target genotype (e.g., the normal or wild type gene rather than the mutant gene) are selected for further development.

In this example, the mutant gene associated with sickle cell anemia is detected by allele-specific hybridization, or “ASH.” This technology is based on the stable annealing of a short, single-stranded oligonucleotide probe to a single-stranded target nucleic acid only when base pairing is completely complementary. The hybridization can then be detected from a radioactive or non-radioactive label on the probe (methods of labeling probes and other nucleic acids are set forth in detail below). ASH markers are used as dominant markers where the presence or absence of only one allele (or haplotype) is determined from hybridization or lack of hybridization by only one probe. The alternative allele may be inferred from the lack of hybridization.

ASH markers have been developed to diagnose susceptibility to human diseases caused by point mutations in DNA sequence. An ASH marker useful in detecting the β^{S}-globin allele associated with sickle-cell anemia is described by Conner et al., Proc. Natl. Acad. Sci. USA, 80:278-282, 1983 and incorporated by reference herein.

Cell samples are taken from each HS population and genomic DNA is extracted using conventional methodology. Following the procedures outlined by Conner et al., supra, the genomic DNA is digested with restriction endonucleases. The resulting nucleic acid fragments are amplified using PCR (see Mullis, K. B. et al., Methods Enzymol. 155:335-350 (1987) incorporated by reference herein) to create amplicons. The amplicons are then transferred to a membrane having a radio-labeled oligonucleotide probe specific for sickle cell anemia bound thereto in a dot-blot format. Hybridization dots are detected by autoradiography. Alternatively, the amplicons can be labeled, and the probes membrane bound.

Once a sample is determined to have the target immunotype and genotype, it may then be differentiated in vivo or in vitro, using the techniques described in U.S. patent application Ser. Nos. 09/997,240, and 10/032,495 incorporated by reference herein, and transplanted into the intended recipient.

For male recipients and female recipient who are not suitable for self-donors due to medical conditions, HS cells having both the target immunotype and genotypes can be derived from female family member donors. Once a sample population is determined to have the target immunotype and genotype, it may then be differentiated in vivo or in vitro, using the techniques described in U.S. patent application Ser. Nos. 09/997,240, and 10/032,495 incorporated by reference herein, and transplanted into the intended recipient.

### Example 4

Construction of a HS cDNA Library

Sampling of Differentiating HS cells. Sections of 5-10 um are cut from the differentiating HS cell system, and frozen immediately −70° C. every few hours, days, or weeks depending on the type of tissue the cells are differentiating into.

Immediately before microdissection (<5 h), cut sections are allowed to warm at room temperature for approximately 1 minute and placed on uncoated glass slides. After removing the layer of glycerol/water from the slide, the cells are microdissected by placing a sterile 30 gauge needle on a pencil sized syringe, and then scraping the cell population of interest while viewing the procedure under the microscope. Following microdissection, the tip of the needle is placed into a small microtube with at least 10 μl solution. Shaking the tube detaches the tissue sample from the needle, and subsequently injecting an air bubble suspends the tissue into the solution, and removes any remaining fragments.

RNA Isolation and cDNA Synthesis. Live differentiating HS cells are harvested at 6 hr intervals, since initiation of differentiation. Total RNA is isolated from these cells as described in Chirgwin et al., Biochemistry (1970) 18:5294-5299, and Hogan et al., supra at 328-330. Total RNA is passed over an oligo-dT cellulose column to obtain polyadenylated RNA, which is stored as an ethanol precipitate at −20° C.

Single and double stranded cDNAs are synthesized from poly A-RNA by modification of the Gubler and Hoffman method, Gubler et al., Gene (1983) 25:263-269, as described below. Following T-4 polymerase blunting and methylation of the cDNAs, synthetic EcoRI linkers are blunt-end ligated. After digestion with EcoRI, the excess linkers are separated from the cDNAs by low-melt agarose gel electrophoresis. Only cDNAs greater than about 650 bp in length are isolated from the low melt gel.

Construction and Screening of a Lambda Phage (lambda-qtl) Library. Purified cDNAs are ligated into EcoRI-digested lambda-gtl. The DNA is then packaged using lambda phage extracts (Gigapack Plus Kit, Strategene). Several fractions of the packaged library are titered in E. coli Y1088 cells; these fractions range from 71% to 81% recombinant phage, as determined by the lack of IPTG-inducible beta-galactosidase activity. The total number of recombinant phage should equal approximately 2.1×10⁶ pfu (plaque forming units). The primary library is then amplified in E. coli Y1088 cells and stored in 7% DMSO at −80° C. The titer of the amplified library should equal approximately 2.5×10⁷ pfu/ml, and further, it should be approximately 65% recombinant.

Two 17-base oligonucleotide probes are synthesized based on amino acid sequence data from isolated peptides derived from protease digested, native polypeptide. The probes are end-labeled with T-4 polynucleotide kinase to high specific activity. E. coli Y1088 cells are infected with enough phage to give 3×10⁴ pfu/plate. The infected cells are plated in 6 ml of top agarose onto 150 mm diameter Luria plates containing 50 μg/ml ampicillin. After overnight incubation at 37° C., the plates are chilled at 4° C. before performing plaque lifts. To eliminate false positive signals, duplicate nitrocellulose filter plaque replicas are prepared from each master plate. Filters are processed by base treatment followed by neutralization in Tris buffer.

The filters are air dried and baked at 80° C. in vacuo. Prehybridization is performed for at least 6 hours 37° C. in 6×SSC, 50 mM Sodium Phosphate (pH 6.8), 5× Denhardt's, and 100 ug/ml denatured Herring sperm prehybridization solution with the addition of dextran sulfate to a final concentration of 10%. The labeled probes are added to the hybridization solution at 1-2×10⁶ cpm/ml.

Filter washes are done in the presence of tetramethylammonium chloride under the conditions described for a 17-base oligonucleotide in Wood et al., Proc. Nat Acad. Sci. USA, (1985) 82:1585-1588, incorporated by reference herein. Each duplicate filter is hybridized to both probes in the first round of screening; in subsequent rounds, the duplicate filters are hybridized to either Probe 1 or Probe 2. All cDNA clones are plaque purified after three or four rounds of screening; phage DNA is isolated from each clone on glycerol step gradients as described in Grossberger, D., Nuc. Acid. Res., (1987)15(16):6737, incorporated by reference herein.

DNA Sequence Analysis. All DNA sequence analysis is done in the M13 vectors mp18 and mp19. Single stranded templates are prepared, and dideoxynucleotide sequencing is preformed using a Sequenase™ DNA Sequencing Kit obtained from United States Biochemical Corporation. Sequencing reactions are primed using either the M13 universal primer, a primer that hybridizes to extraneous lambda-gtil DNA present in some constructs, or the oligonucleotide probes. Sequence data obtained from both ends of the cDNA is analyzed for six base restriction enzyme sites that are used to generate sequencing subclones. In this way, an entire cDNA fragment is sequenced on both strands. All DNA sequences and translated protein sequences are assembled and analyzed using MicroGenie Sequence Software purchased from Beckman.

Expression in E. coli. An initial cDNA clone, lambda-poly-1, is present in the expression vector lambda-gtll. The clone is amplified in E. coli Y1088 cells, and the high titer stock is used to make lysogens in E. coli Y1089. The lambda-poly-1 lysogen is then grown in Luria broth plus ampicillin (50 ug/ml) at 37° C. The cells are pelleted, resuspended in TE buffer, and lysed with lysozyme (2 mg/ml). The cell debris is then pelleted, and the supernatant assayed for activity. Inserts that include a 1 kb lambda-gtil-lacZ DNA attached to the 3′ end, are isolated on a low-melt gel and subcloned into the EcoRI/Sstl sites of pTZ18R (Pharmacia). This construct, pTZpoly-1, is used in the expression and purification of recombinant polypeptides.

Electrophoretic and Western Analysis. Recombinant polypeptide samples are characterized on Commassie-stained SDS-PAGE gels. For Western analysis, the gels are run and transferred to nitrocellulose filters at 30 mA in transfer buffer as described in Burnett, N. W., Analytical Biochemistry (1981) 112:195-203. The filters are blocked with 3% BSA and incubated with a 1/1000 dilution of polyclonal rabbit-anti-luciferase antibodies. Next, the filter is washed in TBS and incubated with al/2500 dilution of the secondary antibody, goat-anti-rabbit IgG conjugated to horseradish peroxidase (Bio-Rad). Finally, the filter was washed in TBS and developed with HRP-Color Developing reagent (Bio-Rad).

### Example 5

Screening and Expression of cDNA in Standard Vectors

Isolation and Analysis of a Recombinant Protein in Lambda phage Vector. A primary screen of 1,000,000 recombinant phage results in the isolation of approximately ten clones, which give identical autoradiographic signals on two replica filters. Of the ten original positives, only five give signals on a second screening, and only one hybridizes to a second probe. The other five hybridize only to a probe with high sequence redundancy.

Restriction enzyme analysis of the five clones that give signal on a second screening is then conducted using known techniques. It is expected that only one recombinant phage hybridizes to the two oligonucleotide probes used. Further said recombinant phage contains cDNA of the target size and is chosen for DNA sequence analysis.

Restriction fragments that contain a 1 kb lambda-gtll-lac-Z DNA fragment are subcloned into M13 and mp18 and mp19 vectors. Both strands of the restriction fragment are then completely sequenced.

Expression of a recombinant polypeptide in E. Coli. A construct, pTZpoly-1, is constructed to simplify the isolation of DNA fragments for use as probes in Southern and Northern analyses. E. coli cells harboring these plasmid are referred to as pTZpoly-1 cells. The pTZ series “phagemids” contain a polylinker site adjacent to the lac Z′ gene. Expressed genes in this vector could potentially be expressed containing the first 10 to 15 amino acids of beta-galactosidase fused to the cDNA translation product. pTZpoly-1 cell supernatants are then analysed for enzyme activity. The pTZpoly-1 crude supernatants are further characterized by SDS-PAGE. Western analysis is performed using rabbit polyclonal antibodies raised against native the native peptide.

Protein Purification. Purification of recombinant polypeptides from pTZ/E. Coli extracts is accomplished in three chromatographic steps. Recombinant polypeptide is purified from pTZpoly-1 cells as follows: pTZpoly-1 cells are grown in 20L Luria broth at 37° C., at an OD₆₀₀=0.6 at which time IPTG is added to a final concentration of 0.5 mM; cells continued to grow overnight at 30° C. The cells are harvested by centrifugation, washed in TE, resuspended in 5 ml of 10 mM EDTA (pH 8) per gram of cells, and frozen at −20° C. In a typical purification, 15 to 30 grams of cells are thawed. Lysozyme is added to a final concentration of 4 to 6 mg/ml, and the cells are held on ice for 45 minutes. DNase 1 (10 to 20 mg) is added to the lysate, which is sonicated on ice with 1 minute bursts from a Branson Cell Disrupter until 90% of the cells are lysed as evidenced by microscopic examination.

The crude material is clarified by centrifugation at 48×g for 30 minutes and loaded onto the first column. The extract is first run on a DEAE-Cellulose ion-exchange column, followed by a G-100 Sephadex gel filtration column, and then a Benzoic Acid-Sepharose affinity column. The G-100 column is run in 1× Standard Buffer (1.5 mM Tris, 1.0 mM EDTA pH 7.8). The other columns are run in 1× buffer and eluted in 10× buffer (DEAE) or Sodium Benzoate in 10× buffer (Benzoic Acid-Sepharose). The first Benzoic Acid column is eluted with 0.1 M sodium benzoate pulse. The second Benzoic Acid column is eluted with a 0 to 0.5 M sodium benzoate gradient. Protein determinations ate made by A₂₈₀ measurements using the extinction coefficient of the native peptide, or by Bradford assays as described in Bradford, M., Analytical Biochemistry (1976) 72: 248.

### Example 6

Proliferation of T-cell Precursor Cells in Response to Cytokine Production

To study the effect of cytokines on differentiation of cultured HS cells into T-cells, in the presence of bone-marrow-derived progenitor cells, using a biocompatible, inorganic matrix the following procedure is employed.

Isolated homozygous stem cells are produced and cultured using techniques described in the foregoing sections. HS cells are then seeded onto tantalum-coated carbon matrices Cell foam (Cytomatrix, Woburn, Mass., USA)) of five different dimensions (5×1 mm, 5×2 mm, 10×1 mm, 10×2 mm, and 30×2 mm), and three different pore densities, and then cultured in 24-well tissue culture plates. A suitable culture medium, comprising Iscove's modified Dulbecco medium (IMDM; Mediatech, Washington, D.C.), 10% Fetal Calf Serum (FCS; Sigma, St. Louis, Mo., USA), glutamine (1 mM), penicillin (10 IU ml⁻¹), and streptomycin (10 IU ml⁻¹), is then added such that the matrices are totally immersed. The culture solution is changed every four days, and all cultures are incubated at 37° C. in 5% carbon dioxide. When the cultures are 80% confluent, as determined by phase-contrast light microscopy, they are transferred to fresh culture plates, cultured for an addition 24 hours.

Progenitor cells for the desired tissue, cell type, or group of cells being studied, are then added to the HS-cell culture. For example, to generate mature T-cells, human bone marrow-derived AC133 or CD34 progenitor cells may be used. Bone marrow is obtained posterior iliac crest aspiration from healthy adult volunteers. Following gradient-density centrifugation, AC133 cells are isolated, using an AC133 Cell Isolation kit (Biotech Inc., Piscataway, N.J., USA). Progenitor cells are added to the HS cell cultures at cell densities of 100,000, 10,000, and 1,000 cells per well. Nonadherent cells are aspirated at 4, 7, 10, 14, and 21 days, and are assessed and counted by flow-cytomethry flourochrome-congugated antibodies.

Proliferative responses of T-cells generated in the co-culture are measured using 3.0-3.5×10³ cells ml⁻¹ with 10 IU ml⁻¹ IL-2 and 2 μg ml⁻¹ PHA in 24-well plates. After seven days the cells are counted and analyzed using flow cytometry. Cytokine production is evaluated in T-cells stimulated with Con A (Sigma; final concentration 5 μg ml⁻¹), treated with Brefeldin A (Sigma).

Cells are harvested or sampled using techniques described in the foregoing examples, and stained with anti-CD4, and anti-CD8 Followed by intracellular staining with TNF-α. Harvested cells are then used to extract and isolate mRNA to generate polypeptides and a cDNA library using the methodology described in the foregoing example.

### Example 7

Artificial Kidney Support Systems

The isolated homozygous (or teratoma) stem cells of the present invention can be used to construct artifical kidney support systems that are alternatives to kidney dialyzers currently available. To create an artifical kidney support system, isolated HS cells are produced and cultured using techniques described in the foregoing sections. Totipotent or unipotent HS cells are then transplanted under the kidney capsule in a recipient nude mouse to grow teratoma stem cells under the influence of endogenous factors.

Transplantation procedure: A nude recipient mouse is anesthetized intraperitoneally with 0.015-0.017 ml of 2.5% avertin per gram of body weight. To expose the capsule, a 1-cm long incision is made in the abdomen of the recipient mouse, after wiping the back with 70% ethanol. The abdominal incision is slid to one side, and another incision is then made directly above the level of the ovary to cut the body wall. Blunt fine forceps are then used to pull out the kidney by its fat pad, to be immobilized. The kidney surface is allowed to dry, and using watchmaker's forceps, a small tear is made in the capsule membrane. The membrane is moistened with sterile saline or PBS. A pocket is then made underneath the capsule, and totipotent stem cell populations can then be inserted into the pocket to differentiate in vivo.

Construction of the Artificial Kidney Support System. Differentiated primary cells are extracted from the kidney capsule, and suspended in low glucose Dulbecco's MEM medium (25 mM HEPES, 4 mM L-glutamine, 5.5 mM glucose, and 5% fetal calf serum) at 37° C. to obtain an initial cell density of 1×10⁵ cells/mL⁻¹.

The cells are then cultivated in a 40 ml fixed-bed unit over a period of about 10 weeks, or until a cell density of 8.5×10⁷ cells/mL⁻¹ is reached. Cultivation is accomplished by immobilizing cells on a macroporous membrane (cellulose based) within the fixed-bed unit, while pumping medium continuously from a conditioning vessel to the fixed-bed matrix and back. The conditioning vessel has a working volume of 500 ml, and is connected to the fixed-bed unit by means of a looped tube. Moreover, the fixed-bed unit is removably arranged at the center of the conditioning vessel.

For initial inoculation of the fixed-bed unit with the cells, the connection between the conditioning vessel and the fixed-bed unit is closed, and a connection between the fixed-bed unit and an inoculation bottle is opened. Suspended cells from an inoculation bottle are pumped through the fixed-bed unit for about two hours. Once inoculation is complete, the connection between the conditioning vessel and the fixed-bed is re-opened. Oxygen-enriched medium is then pumped from the conditioning vessel to the fixed-bed unit and then back to the conditioning vessel. While cells are retained in the fixed-bed unit, spend medium is continuously harvested from the conditioning vessel. The medium used is Dulbecco's MEM with higher concentrations of glucose than the original growth medium. Moreover, the medium is mixed and oxygenated by aeration with air or oxygen. pH of the medium is controlled by the addition of carbon dioxide.

The disclosure of each publication, patent, or patent application mentioned in this specification is incorporated by reference in its entirety to the same extent as if each individual publication, patent, or patent application were specifically and individually indicated to be incorporated by reference.

Although the foregoing invention has been described in some detail by way of illustration and example, for purposes of clarity and understanding, it will be apparent to those skilled in the art that certain changes and modifications are within the scope of the appended claims.

What is claimed is:

1. A method for constructing a model of cellular development and differentiation using homozygous stem cells derived from donor material comprising the steps of:
(a) creating isolated homozygous stem (HS) cells;
(b) producing a desired cell, group of cells, or tissue type by directing differentiation of said isolated HS cells; and,
(c) periodically sampling said isolated HS cells undergoing directed differentiation;

2. A method of claim 1, wherein periodic sampling of isolated HS cells undergoing directed differentiation comprises:
(a) intermittently extracting cellular RNA, and then isolating mRNA from HS cells undergoing directed differentiation; and,
(b) constructing a cDNA library from said isolated mRNA.

3. The method of claim 1, wherein said HS cell of step (a) is created from donor germ material by:
(a) producing a mitotically activated homozygous post-meiosis I diploid germ cell by: fusing two oocytes or two spermatids, preventing the extrusion of the second polar body during oogenesis, allowing the extrusion of the second polar body and spontaneous self-replication under appropriate conditions, or transferring two sperm or two haploid egg nuclei into an enucleated oocyte;
(b) culturing said activated homozygous post-meiosis I diploid germ cell to form a blastocyst-like mass; and,
(c) isolating homozygous stem cells from the inner cell mass of said blastocyst-like mass,
wherein, when a mitotically activated post-meiosis I diploid germ cell is produced by fusing two oocytes or two spermatids, or transferring two sperm or two haploid egg nuclei into an enucleated oocyte, the homozygosity of the isolated stem cells is confirmed by genotyping.

4. The method of claim 1, wherein the differentiation of step (b) is accomplished in vitro by the inclusion of a cell regulating factor, hormone or cytokine in the culture medium.

5. The method of claim 1, wherein the differentiation of step (b) is accomplished in vivo, and comprises transplanting HS cells into a kidney capsule, peritoneal cavity, or subcutaneously.

6. The method of claim 1, wherein the differentiation of step (b) is accomplished using a three-dimensional culture system comprising seeding precurser support cells that are tissue-specific onto a nylon screen, and then inoculating said nylon screen with fresh or cryopreserved HS cells.

7. The method of claim 1, wherein the differentiation of step (b) is accomplished using a three-dimensional culture system comprising:
(a) seeding of HS cells onto a biodegradable and
(b) transplanting said mesh into a vehicle or a microenvironment containing cell growth regulators, hormones, and/or cytokines.

8. The method of claim 7, wherein said microenvironment is a kidney capsule.

9. The method of claim 7, wherein said microenvironment is a peritoneal cavity.

10. The method of claim 7, wherein said microenvironment is HS cells grown with different tissue elements in vitro.

11. The method of claim 7, wherein said vehicle is a nude mouse.

12. The method of claim 1, wherein the differentiation of step (b) is accomplished in vivo and in vitro, comprising:
(a) transplanting HS cells to a microenvironment;
(b) isolating progenitor cells from said microenvironment, and,
(c) further differentiating said progenitor cells in vitro.

13. The method of claim 1, wherein said sampling of step (c) comprises harvesting HS cells at various stages of in vitro differentiation.

14. The method of claim 1, wherein said sampling of step (c) comprises microscopic identification and microdissection of HS cells at various stages of in vivo differentiation, or in vitro differentiation within a three-dimensional culture.

15. The method of claim 1, wherein the desired group of cells are keratinizing epithelial cells.

16. The method of claim 15, wherein said keratinizing epithelial cells are selected from the group consisting of keratinocytes of the epidermis, basal cells of the epidermis, keratinocytes of fingernails and toenails, basal cells of nail bed, hair shaft cells, hair-root sheath cells, and hair matrix cells.

17. The method of claim 1, wherein the desired group of cells is cells of wet stratified barrier epithelia.

18. The method of claim 1, wherein the desired group of cells is epithelial cells specialized for exocrine secretion.

19. The method of claim 1, wherein the desired group of cells is cells specialized for secretion of hormones.

20. The method of claim 1, wherein the desired group of cells is epithelial absorptive cells of the gut, exocrine glands, and urogenital tract.

21. The method of claim 1, wherein the desired group of cells is cells specialized for metabolism and storage.

22. The method of claim 1, wherein the desired group of cells is barrier epithelial cells that line the lungs, gut, exocrine glands, and urogenital tract.

23. The method of claim 1, wherein the desired group of cells is epithelial cells lining closed internal body cavities.

24. The method of claim 1, wherein the desired group of cells is ciliated cells with propulsive function.

25. The method of claim 1, wherein the desired group of cells is cells specialized for secretion of extracellular matrix.

26. The method of claim 1, wherein the desired group of cells is contractile cells.

27. The method of claim 1, wherein the desired group of cells are cells of the blood and immune system.

28. The method of claim 1, wherein the desired group of cells is sensory transducers.

29. The method of claim 1, wherein the desired group of cells is autonomic neurons.

30. The method of claim 1, wherein the desired group of cells is supporting cells of sense organs and of peripheral neurons.

31. The method of claim 1, wherein the desired group of cells is cells of central nervous system comprising neurons and glial cells.

32. The method of claim 1, wherein the desired group of cells is lens cells.

33. The method of claim 1, wherein the desired group of cells is pigment cells.

34. The method of claim 1, wherein the desired group of cells is germ cells.

35. The method of claim 1, wherein the desired group of cells is nurse cells.

36. Isolated mRNA, comprising nucleotide sequences encoding various polypeptides, wherein said mRNA is extracted and isolated at appropriate time intervals from isolated HS cells at various stages of directed differentiation in vivo, and/or in vitro.

37. The mRNA of claim 36, wherein said nucleotide sequences are preceded by a functional promotor sequence 5′ to said sequence.

38. The mRNA of claim 36, wherein at least one copy of said nucleotide sequences is present in a recombinant RNA vector.

39. Isolated cDNA, comprising nucleotide sequences complementary to isolated mRNA, wherein said mRNA is extracted and isolated at appropriate time intervals from isolated HS cells at various stages of directed differentiation in vivo, and/or in vitro.

40. The cDNA of claim 39, wherein said nucleotide sequences are preceded by a functional promotor sequence 5′ to said sequence.

41. The cDNA of claim 39, wherein at least one copy of said nucleotide sequences is present in a recombinant DNA vector.

42. Isolated oligonucleotides complementary to mRNA, wherein said mRNA is extracted and isolated from isolated HS cells at various stages of directed differentiation, and wherein said isolated oligonucleotides comprise at least 10 consecutive nucleotides having at least 65% homology to said mRNA.

43. Isolated oligonucleotides complementary to cDNA, wherein said cDNA is complementary to mRNA molecules extracted and isolated from isolated HS cells at various stages of directed differentiation, and wherein said isolated oligonucleotides comprise at least 10 consecutive nucleotides having at least 65% homology to said cDNA.

44. The isolated oligonucleotides of claim 42 and 43, wherein said oligonucleotides are labeled with a detectable tag.

45. Isolated peptides, polypeptides, and proteins encoded by mRNA extracted and isolated from isolated HS cells at various stages of directed differentiation.

46. A method of identifying genetic material encoding various genes involved in the differentiation of isolated HS cells into a desired cell, group of cells, or tissue type, comprising,
(a) isolating genetic material from differentiating HS cells to form a sample of genetic material,
(b) contacting said sample with an oligonucleotide under hybridizing conditions, and,
(c) detecting the formation of a duplex comprising said oligonucleotide and said genetic material present in said sample.

47. A method for testing the effect of a stimulus on cellular differentiation using homozygous stem (HS) cell systems derived from donor material, comprising,
(a) contacting the stimulus with isolated HS cells at various stages of directed differentiation; and,
(b) sampling said HS cells at various intervals.

48. A method for detecting the effect of a stimulus on cellular differentiation by contacting said stimulus with homozygous stem (HS) cells derived from donor material, comprising,
(a) periodically extracting cellular RNA from HS cell in contact with said stimulus;
(b) isolating mRNA from said cellular RNA; and,
(c) constructing a cDNA library from said isolated mRNA.

49. A method of providing an electronic database of gene sequences and/or proteins involved in cellular development and differentiation of homozygous stem cells, comprising:
(a) receiving gene and protein sequence data to be stored at a remote location for subsequent search and retrieval;
(b) executing a customized search at a locate computer using a key field entry;
(c) transferring the search query of step (b) to said remote location and
retrieving hits, comprising genes and proteins sequences corresponding to said search query within specified error tolerance; and,
(d) presenting hit data in a graphical and pictorial format.

50. An internet based database system for gene sequences and/or proteins involved in cellular development and differentiation of homozygous stem cells, comprising:
(a) a means for accessing and storing information;
(b) a means for formulating a customized search query;
(c) a means for executing the customized search query; and
(d) a means for returning results from the search query in a specified pictorial and graphical layout; wherein the search request can be performed on a plurality of key field.

51. An artificial organ support system that comprises,
(a) a fixed bed, comprising tissue cells immobilized on a macroporous carrier, wherein said tissue cells are cultured by directed differentiation of isolated HS cells, and are capable of sustained proliferation; and,
(b) a conditioning vessel connected to said fixed bed, wherein growth medium is pumped from said conditioning vessel to said fixed bed, and back.

52. An artificial organ support system of claim 51, wherein said organ is a kidney, and said tissue cells are selected from the group comprising of parietal cells and podocytes of the glomerulus, cells of the thin segment of the loop of Henle, duct cells, cells of the juxtaglomerular apparatus, brush border cells of the proximal tubule, and distal tubule cells.

53. An artificial organ support system of claim 51, wherein said organ is a pancreas, and said tissue cells are chosen from a group consisting of alpha, beta, and delta cells of the Islets of Langerhans.

54. An artificial organ support system of claim 51, wherein said organ is a liver, and said tissue cells are hepatocytes.
